(19) United States
(12) Patent Application Publication
(10) Pub. No.: US 2026/0027101 A1
(43) Pub. Date: Jan. 29, 2026

(54) BACTERIAL TOPOISOMERASE INHIBITORS

(71) Applicant: Ohio State Innovation Foundation, Columbus, OH (US)

(72) Inventors: Mark MITTON-FRY, Columbus, OH (US); Joshua POWELL, Columbus, OH (US)

(21) Appl. No.: 18/996,442

(22) PCT Filed: Jul. 19, 2023
(86) PCT No.: PCT/US2023/028100

§ 371 Date: Jan. 17, 2025

Related U.S. Application Data
(60) Provisional application No. 63/390,520, filed on Jul. 19, 2022.

(51) Int. Cl. A61K 31/4545; A61K 31/538; A61P 31/04; C07D 471/04

(52) U.S. Cl. CPC A61K 31/4545; A61K 31/538; A61P 31/04; C07D 471/04

(57) ABSTRACT

Disclosed are bacterial topoisomerase inhibitors with antibacterial activity, including against fluoroquinolone-resistant Staphylococcus aureus, comprising structural domains: a) a left-hand side (LHS) usually comprising a fused bicyclic or tricyclic ring system, b) a linker domain, and c) a right-hand side (RHS) comprising an aromatic or heteroaromatic ring, wherein the novel topoisomerase inhibitors do not contain a secondary amine at the start of the RHS, which is the enzyme-binding moiety.

### CROSS REFERENCE TO RELATED APPLICATIONS

This application claims the benefit of priority to U.S. Provisional Application 63/390,520, filed Jul. 19, 2022, which is incorporated by reference herein in its entirety.

### BACKGROUND

The United States Centers for Disease Control and Prevention (CDC) classified MRSA as a serious threat and ranked it second only to Clostridioides difficile as a source of mortality, with >10,000 deaths per year in the U.S. While the past decade has seen numerous new medicines approved for MRSA infection, morbidity and mortality remain unacceptably high, and new therapeutic options are badly needed.

Novel bacterial topoisomerase inhibitors (NBTIs) (Mitton-Fry, M. J. Novel Bacterial Type II Topoisomerase Inhibitors. Med. Chem. Rev. 2017, 52, 281-302) have emerged as a promising strategy for the cure of infections caused by MRSA and other antibiotic-resistant bacteria. Importantly, however, the NBTIs bind to a novel site (Bax, B. D., et al., Type IIA Topoisomerase Inhibition by a New Class of Antibacterial Agents. Nature 2010, 466, 935-940) and exhibit mechanistic differences from the fluoroquinolones (Bax, B. D., et al., Type IIA Topoisomerase Inhibition by a New Class of Antibacterial Agents. Nature 2010, 466, 935-940; Gibson, E. G., et al., Mechanistic and Structural Basis for the Actions of the Antibacterial Gepotidacin against Staphylococcus aureus Gyrase. ACS Infect. Dis. 2019, 5, 570-581; Gibson, E. G., et al., Bimodal Actions of a Naphthyridinone/Aminopiperidine-Based Antibacterial that Targets Gyrase and Topoisomerase IV. Biochem. 2019, 58, 4447-4455). As a result, the NBTIs lack cross-resistance with fluoroquinolones. Challenges to the success of the NBTI class remain, however, including cardiovascular safety concerns associated with hERG inhibition (Kolarič, A., et al., Novel Bacterial Topoisomerase Inhibitors: Challenges and Perspectives in Reducing hERG Toxicity. Future Med. Chem. 2018, 10, 2241-2244).

Examples of some NBTIs in the literature are provided in Scheme 1. As illustrated by GSK299423 (1) (Bax, B. D., et al. Nature 2010, 466, 935) and summarized by Singh (Singh, S. B., et al. ACS Med. Chem. Lett. 2014, S, 609), NBTIs share three common structural domains: a) a left-hand side (LHS) usually comprising a fused bicyclic or tricyclic ring system, b) a linker domain with an amine positioned to interact with D83 of gyrase, and c) a right-hand side (RHS) comprising an aromatic or heteroaromatic ring. X-ray crystallography has been used to study the binding of these compounds to a complex of gyrase and DNA. This research has provided insight at the molecular level into compound binding (Widdowson, K., et al. Future Med Chem. 2010, 2. 1619; Lahiri, S. D., et al. Antimicrob. Agents Chemother. 2015, 59. 5278), and this understanding has been enhanced through the study of target mutations conferring resistance to NBTIs.

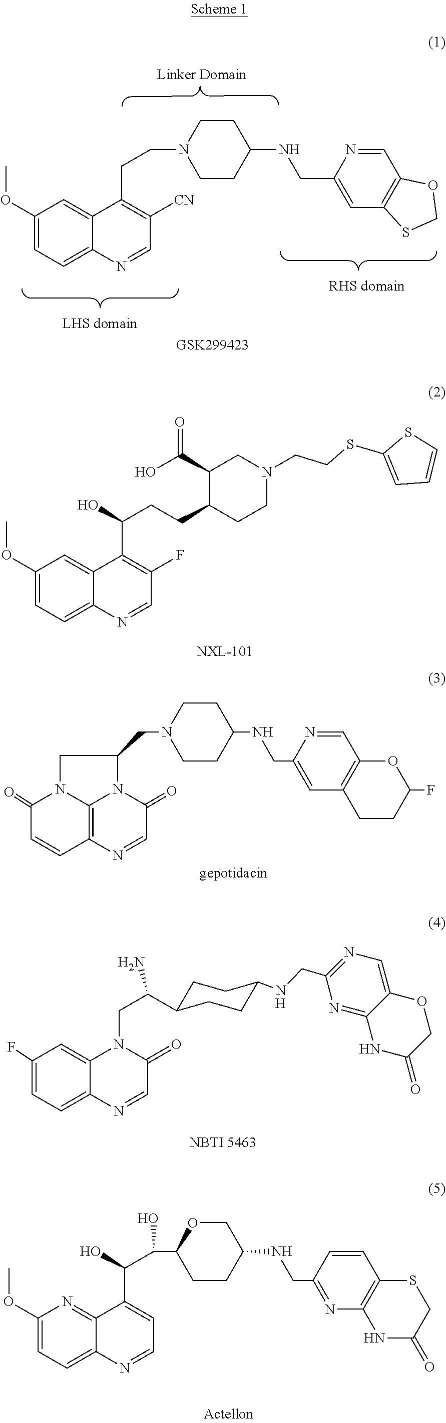

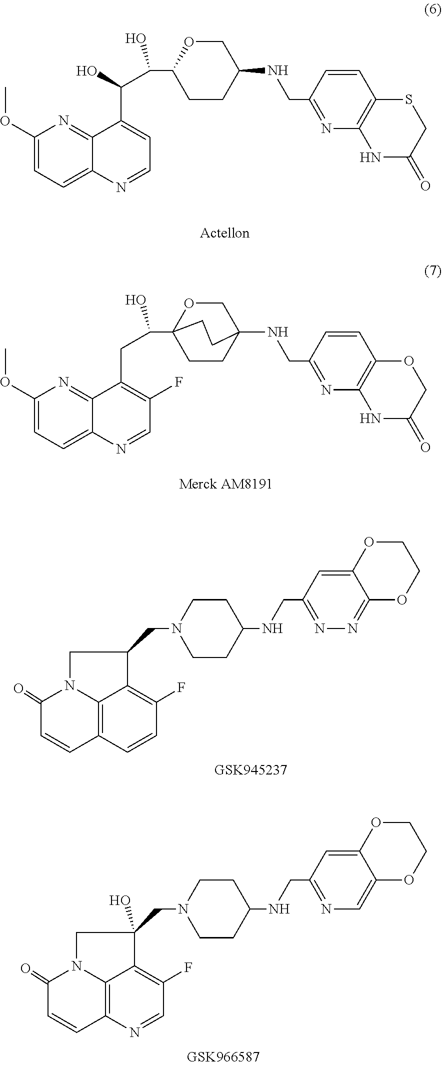 [SMILES: COc1ccc2nccc([C@@H](O)[C@H](O)[C@H]3CC[C@H](NCc4ccc5c(n4)NC(=O)CS5)CO3)c2n1 ; COc1ccc2ncc(F)c(C[C@H](O)C34CCC(NCc5ccc6c(n5)NC(=O)CO6)(CC3)CO4)c2n1 ; O=c1ccc2ccc(F)c3c2n1C[C@H]3CN1CCC(NCc2cc3c(nn2)OCCO3)CC1 ; O=c1ccc2ncc(F)c3c2n1C[C@@]3(O)CN1CCC(NCc2cc3c(cn2)OCCO3)CC1]

All of these examples implicate a secondary amine (or tertiary in the case of NXL-101) as a key binding at the start of the RHS (right hand side, which is the enzyme-binding moiety), since it is involved in binding to gyrase. The position-7 basic amine shows a polar interaction with the Asp83 side chain accounts at least partially for the high affinity of the compounds (Singh, et al. ACS Med. Chem. Lett. 2014, 5, 609).

Disclosed herein are new NBTIs, ones which do not contain the secondary amine thought to be beneficial for binding. The disclosed compounds employ a linker moiety derived from 1,4-substituted piperidine.

### SUMMARY

In accordance with the purposes of the disclosed materials and methods, as embodied and broadly described herein, the disclosed subject matter, in one aspect, relates to compounds, compositions and methods of making and using compounds and compositions. In specific aspects, the disclosed subject matter relates to Novel Bacterial Type II Topoisomerase Inhibitors (NBTIs), analogs thereof, pharmaceutical compositions thereof, and methods of making and using these compounds and compositions. In further aspects, the disclosed subject matter relates to NBTIs with both gyrase and TopoIV activity, analogs thereof, pharmaceutical compositions thereof, and methods of making and using these compounds and compositions. The disclosed compounds can have potent and balanced inhibition of gyrase and TopoIV (to maximize bacterial killing and slow resistance emergence), minimal hERG inhibition (to reduce cardiotoxicity liabilities), and physicochemical properties consistent with desirable pharmacokinetic (PK) properties. Methods of using the disclosed compounds to treat infections, such as MRSA, MDR P. aeruginosa, and other pathogens are also described herein.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

The materials, compounds, compositions, and methods described herein may be understood more readily by reference to the following detailed description of specific aspects of the disclosed subject matter, and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an inhibitor” includes mixtures of two or more such inhibitors, reference to “the kinase” includes mixtures of two or more such kinases, and the like.

“Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

By “reduce” or other forms of the word, such as “reducing” or “reduction,” is meant lowering of an event or characteristic (e.g., bacterial growth or infection). It is understood that this is typically in relation to some standard or expected value, in other words it is relative, but that it is not always necessary for the standard or relative value to be referred to. For example, “reduces bacterial growth” means decreasing the amount of bacteria cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as bacterial growth or infection), diminishment of extent of infection, stabilized (i.e., not worsening) state of infection, preventing or delaying spread of the infection, preventing or delaying occurrence or recurrence of infection, delay or slowing of infection progression, and amelioration of the infected state.

The term “patient” preferably refers to a human in need of treatment for any purpose, and more preferably a human in need of a treatment to treat infection. However, the term “patient” can also refer to non-human animals, preferably mammals such as dogs, cats, horses, cows, pigs, sheep and non-human primates, among others, that are in need of treatment with a compound as disclosed herein.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Chemical Definitions

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms (C₁-C₂₄), such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halo, hydroxy, oxo (i.e., ═O), nitro, silyl, sulfo-oxo, sulfonyl, sulfone, sulfoxide, or thiol, as described below. In specific examples, an alkyl group can be C₁-C₁₈, C₁-C₁₀, or C₁-C₆ alkyl.

The symbols Aⁿ is used herein as merely a generic substituent in the definitions below.

The term “alkoxy” as used herein is an alkyl group, as defined herein, bound through a single, terminal ether linkage; that is, an “alkoxy” group can be defined as —OA¹ where A¹ is alkyl as defined above.

The term “alkenyl” as used herein is a hydrocarbon group of from 2 to 24 carbon atoms with a structural formula containing at least one carbon-carbon double bond (C₂-C₂₄). Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halo, hydroxy, oxo, nitro, silyl, sulfo-oxo, sulfonyl, sulfone, sulfoxide, or thiol, as described below. In specific examples, an alkenyl group can be C₂-C₁₈, C₂-C₁₀, or C₂-C₆ alkenyl.

The term “alkynyl” as used herein is a hydrocarbon group of 2 to 24 carbon atoms with a structural formula containing at least one carbon-carbon triple bond (C₂-C₂₄). The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halo, hydroxy, oxo, nitro, silyl, sulfo-oxo, sulfonyl, sulfone, sulfoxide, or thiol, as described below. In specific examples, an alkynyl group can be C₂-C₁₈, C₂-C₁₀, or C₂-C₆ alkynyl.

The term “aryl” as used herein is a group that contains any carbon-based aromatic group having from 5 to 15 carbon atoms including, but not limited to, benzene, naphthalene, phenyl, biphenyl, phenoxybenzene, and the like. The term “heteroaryl” is defined as a group that contains an aromatic group that has from 4 to 15 carbon atoms and at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl and heteroaryl group can be substituted or unsubstituted. The aryl and heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halo, hydroxy, oxo, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of from 3 to 15 carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halo, hydroxy, oxo, nitro, silyl, sulfo-oxo, sulfonyl, sulfone, sulfoxide, or thiol as described herein.

The term “cycloalkenyl” as used herein is a non-aromatic carbon-based ring composed of from 3 to 15 carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halo, hydroxy, oxo, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for C═O, which is also referred to as oxo.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O⁻.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above. The term “oxo” is used to define ═O.

The term “halo” or “halide” as used herein refers to the halogens fluorine, chlorine, bromine, and iodine.

The term “hydroxyl” as used herein is represented by the formula —OH.

The term “nitro” as used herein is represented by the formula —NO₂.

The term “cyano” as used herein is represented by the formula —CN

The term “azido” as used herein is represented by the formula —N₃.

The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above. The term “sulfoxide” is used herein to refer to the sulfo-oxo group represented by the formula —OS(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH₂.

The term “thiol” as used herein is represented by the formula —SH.

As used herein, the symbol “ [symbol] ” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “ [symbol] ” indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or “ [symbol] ” infers that when R³ is “XY”, the point of attachment bond is the same bond as the bond by which R³ is depicted as being bonded to CH₃.

It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salt” refers to a salt that is pharmaceutically acceptable and has the desired pharmacological properties. Such salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to infection, an effective amount comprises an amount sufficient to cause a bacterial cell to shrink and/or to decrease the growth rate of the bacterial cells or to prevent or delay other unwanted infection. In some embodiments, an effective amount is an amount sufficient to delay development. In some embodiments, an effective amount is an amount sufficient to prevent or delay occurrence and/or recurrence. An effective amount can be administered in one or more doses. In the case of bacterial infection, the effective amount of the drug or composition may: (i) reduce the number of bacterial cells; (ii) reduce bacterial cell size; (iii) inhibit, retard, slow to some extent and preferably stop bacterial cell infiltration into peripheral organs; (iv) inhibit bacterial growth; (vi) prevent or delay occurrence and/or recurrence of bacterial infection; and/or (vii) relieve to some extent one or more of the symptoms associated with the infection.

Effective amounts of a compound or composition described herein for treating a mammalian subject can include about 0.1 to about 1000 mg/Kg of body weight of the subject/day, such as from about 1 to about 100 mg/Kg/day, especially from about 10 to about 100 mg/Kg/day. The doses can be acute or chronic. A broad range of disclosed composition dosages are believed to be both safe and effective.

Reference will now be made in detail to specific aspects of the disclosed materials, compounds, compositions, articles, and methods, examples of which are illustrated in the accompanying Examples.

### Compounds

As illustrated in Scheme 1. The LHS binds with DNA, and the RHS, generally containing an aromatic or heteroaromatic ring, binds to a dimeric interface of gyrase.

Commonly observed gyrase mutations conferring resistance to NBTIs, such as substitutions at D83 and M121, occur at this interface. Extensive previous efforts have optimized the LHS and RHS moieties and illustrated the tolerance for structural variety and innovation in the linker (Surivet, J-P., et al. J. Med. Chem. 2017, 60, 3776; Li, L., et al. Bioorg. Med. Chem. Lett. 2018, 28, 2477; Li, L., et al., ACS Infect. Dis. 2019, 5, 1115; Tan, C. M., et al. Antimicrob. Agents Chemother. 2016, 60, 4830; Black, M. T., et al. Antimicrob. Agents Chemother. 2008, 52. 3339; Mitton-Fry, M. J., et al. Bioorg. Med. Chem. Lett. 2013, 23. 2955; Dougherty, T. J., et al. Antimicrob. Agents Chemother. 2014, 58. 2657; Dougherty, T. J., et al. Antimicrob. Agents Chemother. 2014, 58. 4250; Nayar, A. S., et al. Antimicrob. Agents Chemother. 2015, 59, 331; Reck, F., et al. Boorg. Med. Chem. 2014, 22. 5392; Surivet, J-P., et al. J. Med. Chem. 2013, 56, 7396; Surivet, J-P., et al. J Med. Chem. 2015, 58, 927; Miles, T. J., et al. Bioorg. Med. Chem. Lett. 2011, 21, 7489; Wiles, J. A., et al. J. Med Chem. 2011, 54. 3418; Mitton-Fry, M. J. Novel, Nonquinolone Inhibitors of DNA Gyrase and Topoisomerase IV: Antibacterial Activity and Resistance Mechanisms. Presented at the 243rd National Meeting of the American Chemical Society, San Diego, CA, 2012, Paper MEDI-257; Singh, S. B., et al. Boorg. Med. Chem. Lett. 2015, 25, 2409; Singh, S. B., et al. Bioorg. Med. Chem. Lett. 2015, 25, 3636; Singh, S. B., et al. Med Chem. Commun. 2015, 6, 1773; So, W., et al. Antimicrob. Agents Chemother. 2015, 59. 4956; Miles, T. J., et al. Boorg. Med. Chem. Lett. 2013, 23. 5437).

The largely solvent-exposed linker domain serves to bridge the LHS and RHS and does not itself play a critical role in binding, evidenced by the linker diversity tolerated in compounds in Scheme 1. But as noted, a secondary (or tertiary) amine adjacent to the RHS was a key feature to aid in binding. In the compounds disclosed herein, a new linker moiety is introduced. The structural simplicity and synthetic accessibility of the linker moiety disclosed herein can also result in improved synthetic efficiency and cost effectiveness. Specifically, the disclosed compounds have a 1,4-substituted piperidine linker moiety, shown below.

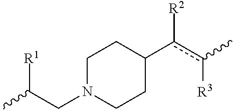

In some aspects, disclosed herein are compounds that are Type II Topoisomerase Inhibitors having Formula I.

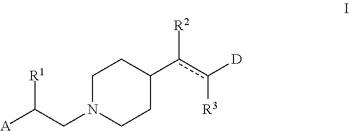

wherein
the dashed line represents a bond that is present or absent, and when the bond is present, R² and R³ are both H;
A is a fused bicyclic aryl or bicyclic heteroaryl ring optionally substituted with C₁-C₂₄ alkyl, C₁-C₂₄ haloalkyl, C₁-C₂₄ alkoxy, C₂-C₂₄ alkenyl, C₂-C₂₄ alkynyl, C₅-C₁₅ aryl, C₄-C₁₅ heteroaryl, aldehyde, amino, amido, carboxylic acid, carboxylic ester, ether, carbamate, halo, hydroxy, oxo, cyano, nitro, silyl, sulfo-oxo, sulfonyl, sulfone, sulfoxide, or thiol; or A and R¹ together form a tricyclic ring optionally substituted with C₁-C₂₄ alkyl, C₁-C₂₄ haloalkyl, C₁-C₂₄ alkoxy, C₂-C₂₄ alkenyl, C₂-C₂₄ alkynyl, C₅-C₁₅ aryl, C₄-C₁₅ heteroaryl, aldehyde, amino, amido, carboxylic acid, carboxylic ester, ether, halo, hydroxy, oxo, cyano, nitro, silyl, sulfo-oxo, sulfonyl, sulfone, sulfoxide, or thiol;
D is an C₅-C₁₅ aryl or C₅-C₁₅ heteroaryl ring optionally substituted with C₁-C₂₄ alkyl, C₁-C₂₄ haloalkyl, C₁-C₂₄ alkoxy, C₂-C₂₄ alkenyl, C₂-C₂₄ alkynyl, C₅-C₁₅ aryl, C₄-C₁₅ heteroaryl, aldehyde, amino, amido, carboxylic acid, carboxylic ester, carbamate, ether, halo, hydroxy, oxo, nitro, silyl, sulfo-oxo, sulfonyl, sulfone, sulfoxide, or thiol;
R¹ is H, OH, or together with A forms a tricyclic ring optionally substituted with C₁-C₂₄ alkyl, C₁-C₂₄ haloalkyl, C₁-C₂₄ alkoxy, C₂-C₂₄ alkenyl, C₂-C₂₄ alkynyl, C₅-C₁₅ aryl, C₄-C₁₅ heteroaryl, aldehyde, amino, amido, carboxylic acid, carboxylic ester, ether, halo, hydroxy, oxo, cyano, nitro, silyl, sulfo-oxo, sulfonyl, sulfone, sulfoxide, or thiol;
R² and R³ are, independently, chosen from H, OH, Cl, F, Br, I, CN, NO₂, NH₂, CF₃, CO₂H, CO₂NH₂, CO₂NHR¹⁰, CO₂R¹⁰, C(O)R¹⁰, C(O)NH₂, C(O)NHR¹⁰, NHC(O)R¹⁰, NHSOR¹⁰, NH, SO₂R¹⁰, oxo, and C₁-C₆ alkyl or C₁-C₆ alkoxyl optionally substituted with C₁-C₂₄ alkoxy, C₂-C₂₄ alkenyl, C₂-C₂₄ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol; or together R² and R³ form a carbamate or carbonate;
R¹⁰ is H, C₁-C₆ alkyl, C₂-C₂₄ alkenyl, C₂-C₂₄ alkynyl, C₅-C₁₅ aryl, or C₄-C₁₅ heteroaryl; or a pharmaceutically acceptable salt thereof.

Formula I contemplates each enantiomer and diastereomer. That is, there can be a chiral center at the carbon attached to R¹, R², and R³, and the present disclosure contemplates each stereoisomer of general Formula I. In some examples, R² is oxo, OH, NH₂ or H. In other examples R³ is H or OH. In still other examples, both R² and R³ are OH. In other examples, R² and R³ are both H and the dashed line is a bond.

In specific examples of Formula I, R¹ is H or OH. In other specific examples, R² and R³ are, independently, chosen from H, OH, and NH₂. In some further examples, R² is NH₂. In another example, R² is H or OH. In another example, R³ is NH₂. In another example, R³ is H or OH. In another example, R² and R³ together form a carbamate or carbonate.

In some preferred examples, disclosed are compounds of Formula 1 where R² and R³ are both OH. In another preferred example, R² is NH₂ and R³ is OH. Still further, R² can be OH and R³ can be NH₂ in Formula 1.

The disclosed compounds can have potent and balanced inhibition of gyrase and TopoIV (to maximize bacterial killing and slow resistance emergence), minimal hERG inhibition (to reduce cardiotoxicity liabilities), and physicochemical properties consistent with desirable pharmacokinetic (PK) properties (Lipinski, C. A., et al. Adv. Drug Delivery Rev. 1997, 23, 3; Veber, D. F., et al. J. Med. Chem. 2002, 45. 2615; Gleeson, M. P. J. Med. Chem. 2008, 51, 817; Leeson, P. D., et al. Nature Rev. Drug Disc. 2007, 6. 881), and ease of synthesis. Further, these compounds can utilize a mechanistically distinct form of topoisomerase inhibition resulting in antibacterial activity even against highly fluoroquinolone-resistant strains.

Previous work with NBTIs has helped to clarify their advantages and remaining challenges. The interactions with the target, distinct from those of fluoroquinolones, lead to a lack of cross-resistance between these two classes of topoisomerase inhibitors (Black, M. T., et al. Antimicrob. Agents Chemother. 2008, 52. 3339; Mitton-Fry, M. J.; Brickner, S. J., et al. Boorg. Med. Chem. Lett. 2013, 23, 2955). As such, NBTIs do not face the issue of widespread preexisting resistance in the clinic that would be encountered with a novel fluoroquinolone. Excellent efficacy against Gram-positive pathogens such as MRSA, both in vitro (Minimum Inhibitory Concentrations, MICs) and in vivo (murine models of infection) has been demonstrated for structurally diverse NBTIs. More recent work has also suggested that an appropriately situated primary amine in the linker domain, such as that found in NBTI 5463 (4, Scheme 1) may be sufficient for antibacterial activity against critically important Gram-negative pathogens such as P. aeruginosa (Dougherty, T. J., et al. Antimicrob. Agents Chemother. 2014, 58, 2657; Dougherty, T. J., et al. Antimicrob. Agents Chemother. 2014, 58. 4250; Nayar, A. S., et al. Antimicrob. Agents Chemother. 2015, 59, 331; see also Surivet, J-P., et al. J. Med. Chem. 2017, 60, 3776) potentially as a result of improved porin penetration. Such effects have also been observed with amine incorporation in other antibacterial drug classes such as cephalosporins.

Among the challenges associated with NBTIs, two deserve special attention. hERG inhibition, with attendant concern about QT-prolongation and cardiovascular safety, must be closely monitored (Li, L., et al., ACS Infect. Dis. 2019, 5, 1115; Kolarič, A., et al., Future Med. Chem. 2018, 10, 2241-2244; Li, L., et al. Bioorg. Med. Chem. Lett. 2018, 28, 2477; Surivet, J-P., et al. J. Med. Chem. 2017, 60, 3776; Reck, F., et al. Bioorg. Med. Chem. 2014, 22. 5392; Miles, T. J., et al. Bioorg. Med. Chem. Lett. 2011, 21. 7483; Geng, B., et al. Boorg. Med. Chem. Lett. 2011, 21. 5432; Reck, F., et al. J. Med. Chem. 2011, 54. 7834; Reck, F., et al. J. Med. Chem. 2012, 55. 6916; Wiles, J. A., et al. J. Med. Chem. 2011, 54. 3418; Singh, S. B., et al. Boorg. Med. Chem. Lett. 2015, 25, 1831; Singh, S. B., et al. Bioorg. Med. Chem. Lett. 2015, 25. 2473). At least one clinical candidate, NXL-101 (2, Scheme 1) (Black, M. T., et al. Antimicrob. Agents Chemother. 2008, 52. 3339) was withdrawn from clinical studies as a result of QT-prolongation. Historically, NBTIs demonstrate superior inhibition of gyrase as compared to TopoIV, at least in S. aureus, permitting resistance by means of single-step mutations to the gyrase target. Improved inhibition of TopoIV has been associated with diminished resistance (Surivet, J-P., et al. J. Med. Chem. 2013, 56, 7396; Surivet, J-P., et al. J. Med. Chem. 2015, 58. 927).

It has been demonstrated that hERG inhibition from NBTIs often correlates strongly with lipophilicity and amine basicity. While not wishing to be bound by theory, the disclosed compounds can minimize hERG inhibition via removal of a basic amine and reduced lipophilicity and provide ready synthetic accessibility across a wide range of derivatives.

Aside from reducing amine basicity and lipophilicity, the readily accessible 1,4-substituted piperidine linker also enhances synthetic efficiency compared to tetrahydropyran (THP) and oxabicyclooctane linkers (Scheme 1). THPs 5, 6, and oxabicylooctane 7 all display excellent antibacterial activity, reinforcing the tolerance for structural changes to the linker, provided that the overall molecular topology is maintained. However, synthesis of the linker alone for 7 required 14 steps, and 5 and 6 suffer from synthetic and stereochemical complexity.

In addition to structural diversity, the disclosed compounds can be used to explore a breadth of physicochemical properties, including CLogP and topological polar surface area (TPSA). Variations in LHS, linker substitution, and RHS can be explored systematically. The LHS plays a key role in interacting with DNA. Quinoline LHS A (Scheme 2) has been used successfully by several teams (Wiles, J. A., et al. J. Med. Chem. 2011, 54, 3418; Mitton-Fry, M. J. Novel, Non-quinolone Inhibitors of DNA Gyrase and Topoisomerase IV: Antibacterial Activity and Resistance Mechanisms. Presented at the 243rd National Meeting of the American Chemical Society, San Diego, CA, 2012, Paper MEDI-257), and 1,5-naphthyridine B (Scheme 2) has likewise seen extensive usage (Li, L., et al., ACS Infect. Dis. 2019, 5, 1115; Singh, S. B., et al. Boorg. Med. Chem. Lett. 2015, 25, 2409; Singh, S. B., et al. Boorg. Med. Chem. Lett. 2015, 25, 3636; Singh. S. B., et al. Med. Chem. Commun. 2015, 6, 1773). LHS C (Scheme 2) dramatically reduces the lipophilicity of the planned analogs (ca. 2 CLogP units versus A) and has been shown to provide potent analogs in several reports. Substitution of the methoxy group of LHS C with fluorine, as in LHS D (Scheme 2), has been shown previously to reduce the undesired inhibition of cardiac ion channels, and D is the core for the promising Gram-negative lead NBTI 5463. Similar evidence for potency has been sought in choosing RHS moieties. The RHS binds to the dimeric gyrase interface and has also been observed to impact target potency against TopoIV. Consequently, variations of the RHS are can be used to improved TopoIV potency and diminished resistance. Moreover, the choice of RHS also appears to impact the degree of inhibition of the hERG and other ion channels. Whereas all of these moieties have been used in potent inhibitors, RHS 2-4, especially RHS 4, have shown reduced hERG inhibition as compared to RHS 5 and RHS 6. Notably, RHS 1 is a key feature of Phase 3 clinical candidate gepotidacin, RHS 2 was used for an earlier candidate GSK966587, and RHS 7 constitutes the RHS of the analog used in breakthrough crystallographic studies. The choice of RHS also enables variation in lipophilicity, hydrogen bond donor/acceptor number, and TPSA.

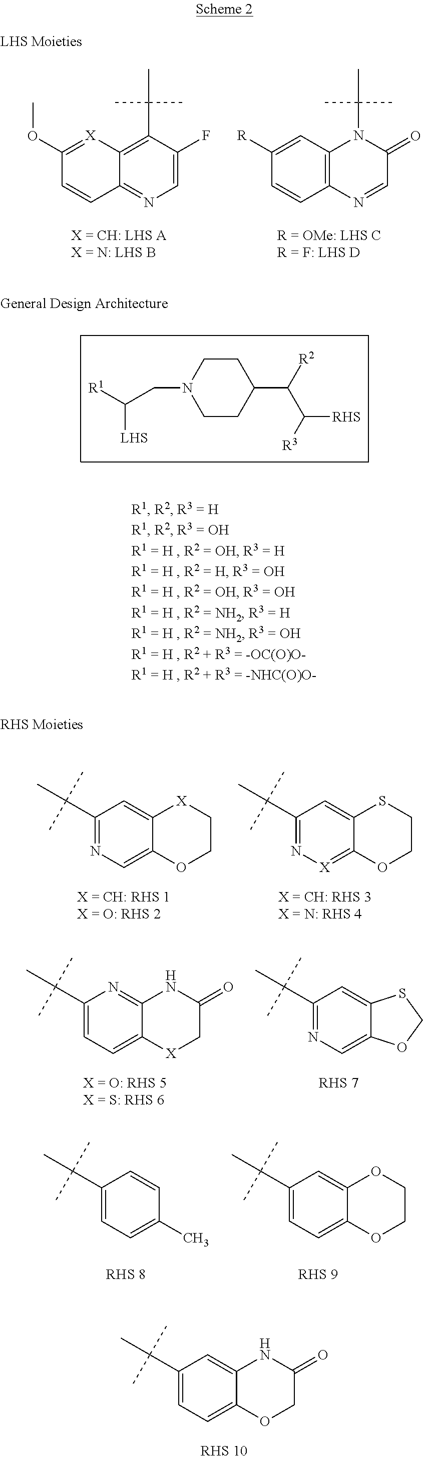

In view of the above, specific examples disclosed herein are compounds of Formula I, wherein A is a fused bicyclic aryl or bicyclic heteroaryl ring having Formula II.

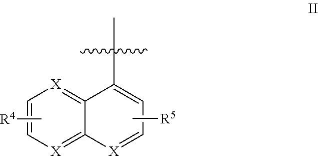

wherein
each X is, independently, CH or N; and
R⁴ and R⁵ are, independently, chosen from H, Cl, F, Br, I, CN, OH, NO₂, NH₂, CF₃, CO₂H, CO₂NH₂, CO₂NHR³, CO₂R³, C(O)R³. C(O)NH₂, C(O)NHR³, and C₁-C₆ alkyl or C₁-C₆ alkoxyl optionally substituted with C₁-C₆ alkyl, C₁-C₆ alkoxyl, C₂-C₆ alkenyl, C₂-C₆ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol.

In specific examples, A can have Formula II, wherein R⁴ and R⁵ are, independently, chosen from H, Cl, F, Br, I, CN, OH, and unsubstituted C₁-C₆ alkyl or C₁-C₆ alkoxyl. In further examples, R⁴ and R³ are, independently, chosen from H, Cl, F, CN, OH, and methoxyl. In further examples, R⁴ and R³ are, independently, chosen from F and methoxyl. In still further examples, all X's are CH. In yet further examples, one X is CH and the other two X's are N. In yet further examples, two X's are CH and the other X is N. In still further examples, all X's are N.

In further examples, disclosed herein are compounds of Formula I, wherein A is a fused bicyclic aryl or bicyclic heteroaryl ring having Formula III.

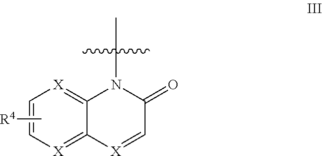

wherein
each X is, independently, CH or N;
R⁴ is chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with C₁-C₆ alkyl, C₁-C₆ alkoxyl, C₂-C₆ alkenyl, C₂-C₆ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol.
   In specific examples, A can have Formula III, wherein R⁴ is chosen from H, Cl, F, Br, I, CN, OH, and unsubstituted C₁-C₆ alkyl or C₁-C₆ alkoxyl. In further examples, R⁴ is chosen from H, Cl, F, CN, OH, and methoxyl. In further examples, R⁴ is chosen from F and methoxyl. In specific examples of Formula III, each X is N. In other examples, two X's are CH and the other X is N. In other examples, two X's are N and the other X is CH.

In still further examples, disclosed herein are compounds of Formula I, wherein A is a bicyclic aryl or bicyclic heteroaryl that together with R¹ forms a tricyclic ring. When R¹ is a CH₂, this can be shown by Formula IX, X, XI, or XII.

In specific examples, A can be Formula IX:

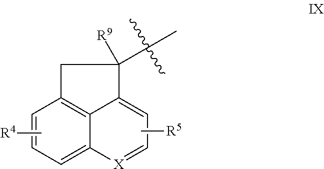

wherein
X is CH, N, or CR⁸.
R⁴ and R⁵ are, independently, chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with C₁-C₆ alkyl, C₁-C₆ alkoxyl, C₂-C₆ alkenyl, C₂-C₆ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol; and
R is Cl, F, CN, OH, OCH₃, CH₃, or NH₂; and
R⁹ is H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, or C₁-C₆ alkyl or C₁-C₆ alkoxyl optionally substituted with C₁-C₆ alkyl, C₁-C₆ alkoxyl, C₂-C₆ alkenyl, C₂-C₆ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol.

In specific examples, A can be Formula X:

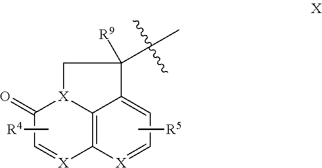

wherein
each X is, independently, CH, N, or CRS;
R⁴ and R⁵ are, independently, chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with C₁-C₆ alkyl, C₁-C₆ alkoxyl, C₂-C₆ alkenyl, C₂-C₆ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol;
R⁸ is, independently, Cl, F, CN, OH, OCH₃, CH₃, or NH₂; and
R⁹ is H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, or C₁-C₆ alkyl or C₁-C₆ alkoxyl optionally substituted with C₁-C₆ alkyl, C₁-C₆ alkoxyl, C₂-C₆ alkenyl, C₂-C₆ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol.

In specific examples, A can be Formula XI:

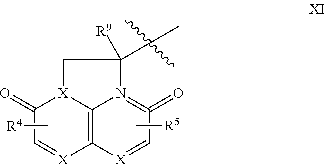

wherein
each X is, independently, CH, N, or CR⁸;
R⁴ and R³ are, independently, chosen from H, Cl, F, Br, I, CN, OH, NO₂, NH₂, CF₃, CO₂H, CO₂NH₂, CO₂NHR³, CO₂R³, C(O)R³. C(O)NH₂, C(O)NHR³, and C₁-C₆ alkyl or C₁-C₆ alkoxyl optionally substituted with C₁-C₆ alkyl, C₁-C₆ alkoxyl, C₂-C₆ alkenyl, C₂-C₆ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol;
each R⁸ is, independently, Cl, F, CN, OH, OCH₃, CH₃, or NH₂; and
R⁹ is H or C₁-C₆ alkyl.

In specific examples, A can be Formula XII:

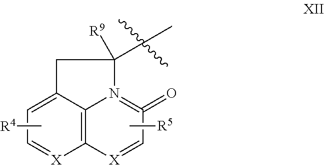

wherein
each X is, independently, CH, N, or CR¹;
R⁴ and R⁵ are, independently, chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with C₁-C₆ alkyl, C₁-C₆ alkoxyl, C₂-C₆ alkenyl, C₂-C₆ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol;
each R⁸ is, independently, Cl, F, CN, OH, OCH₃, CH₃, or NH₂; and
R⁹ is H or C₁-C₆ alkyl.

In specific examples, of Formula I, A can be

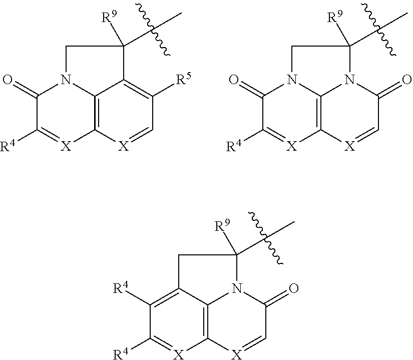

wherein each of R⁴, R⁵, R⁸, and R⁹ can be independently chosen from Cl, F, CN, OH, OCH₃, CH₃, or NH₂. In preferred examples R⁹ is H or CH₃.
   In specific examples, of Formula IX, X, XI, and XII, R⁵ can be F.

In still further examples, disclosed herein are compounds of Formula I, wherein D is aryl or heteroaryl ring having Formula IV-VIII or XIII.

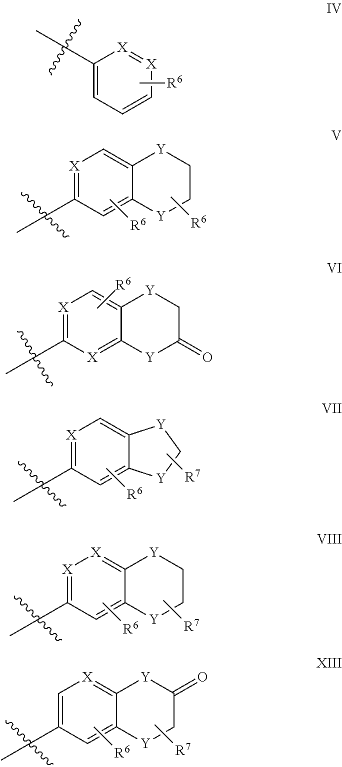

wherein
each X is, independently, chosen from CH and N;
each Y is, independently, chosen from O, S, NH, and CH₂; and
R⁶ and R⁷ are, independently, chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with C₁-C₆ alkyl, C₁-C₆ alkoxyl, C₂-C₆ alkenyl, C₂-C₆ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol.

In specific examples, D can have Formula IV-VIII or XIII, wherein R⁶ and R⁷ are, independently, chosen from H, Cl, F, Br, I, CN, OH, and unsubstituted C₁-C₆ alkyl or C₁-C₆ alkoxyl. In further examples, R⁶ and R⁷ are, independently, chosen from H, Cl, F, CN, OH, and methoxyl. In further examples, R⁶ and R⁷ are, independently, chosen from F and methoxyl. In further examples, R⁶ and R⁷ are both H. In still further examples, both Y are O. In other examples, one Y is S and the other is O. In still other examples, one Y is NH and the other is O.

In yet further examples, disclosed herein are compounds of Formula I, wherein R² and R³ are, independently, chosen from H, F, OH, and NH₂. In specific examples, R² is NH₂. In other examples, R² is H or OH. In further examples, R³ is H or OH. In specific examples, R³ is NH₂. In other examples, R¹ is H. In other examples, R¹ is OH. In further examples, R² and R³ are both H and the dashed line is present, i.e., a double bond is present, which can be cis or trans.

In specific examples, the compounds can have one of the following formulas:

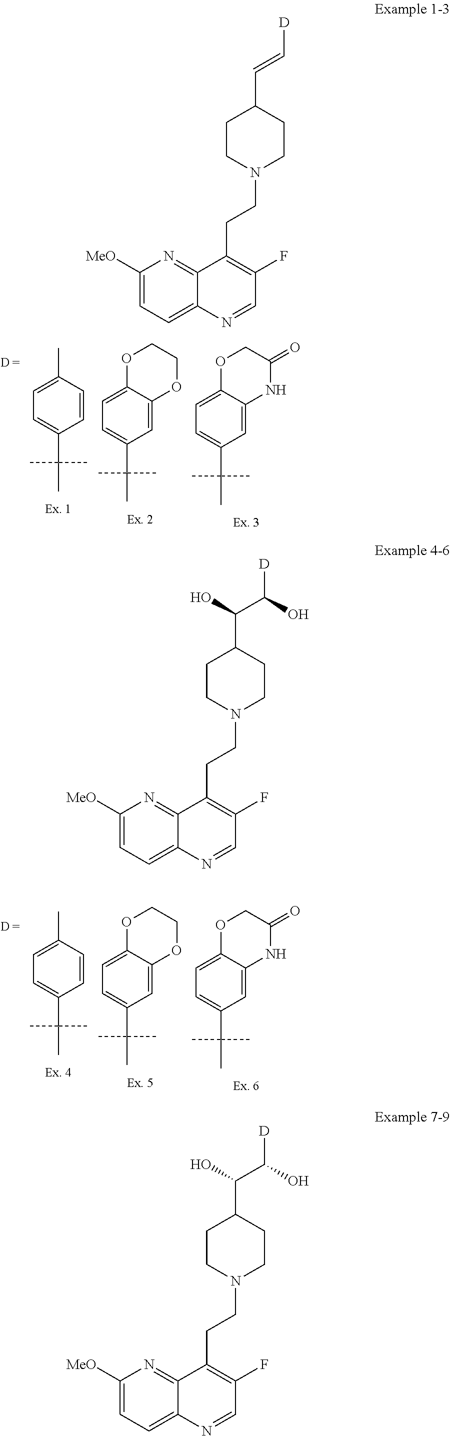

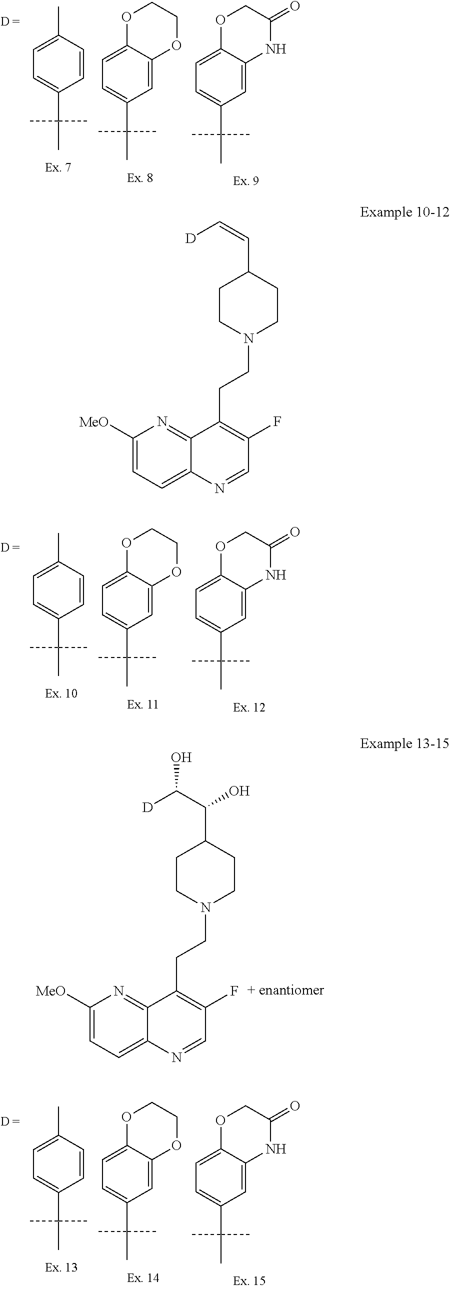

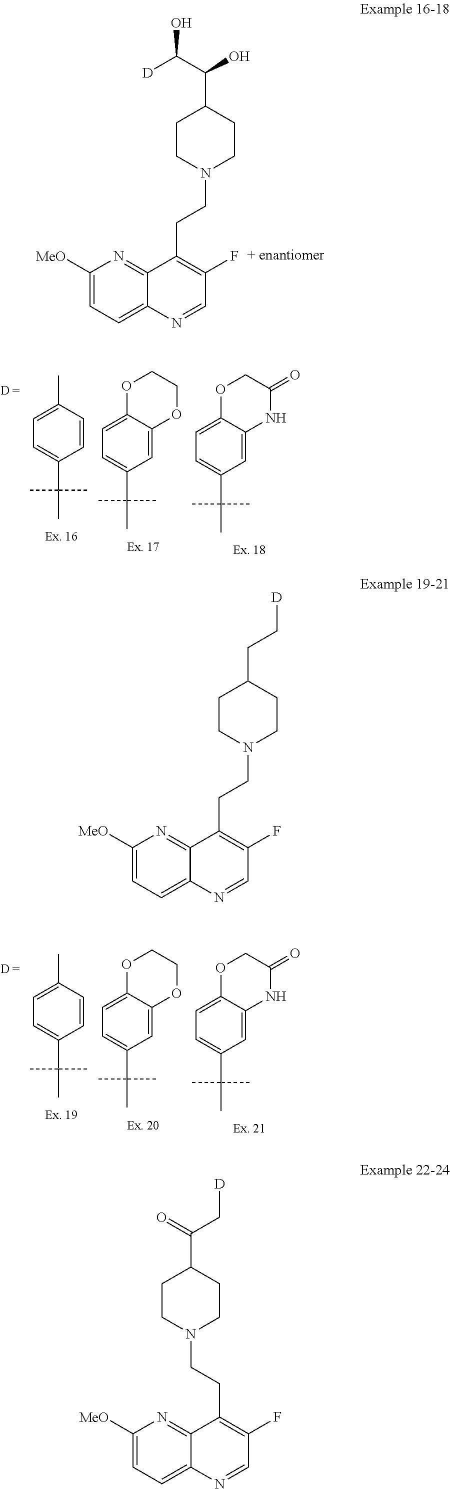

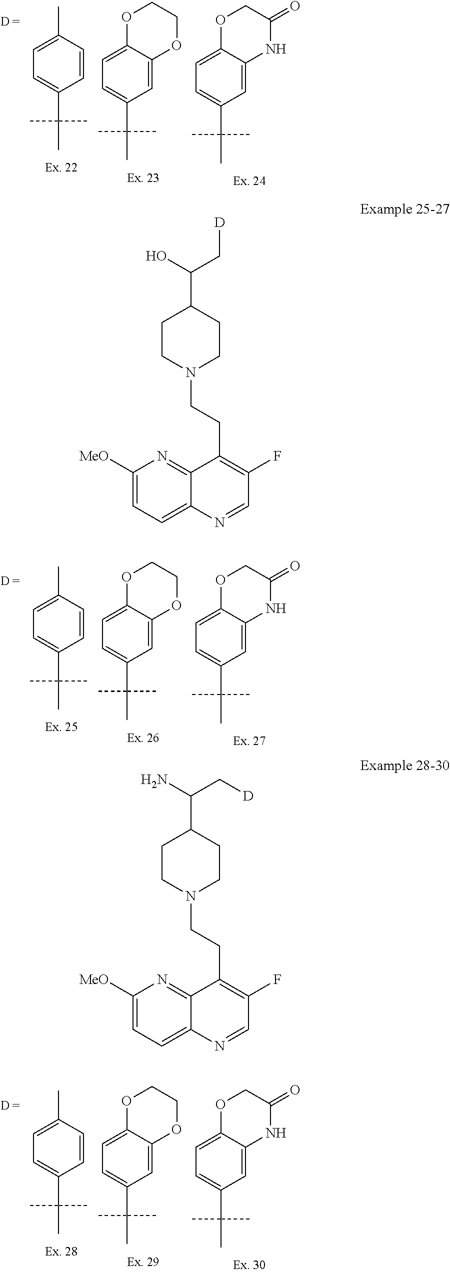

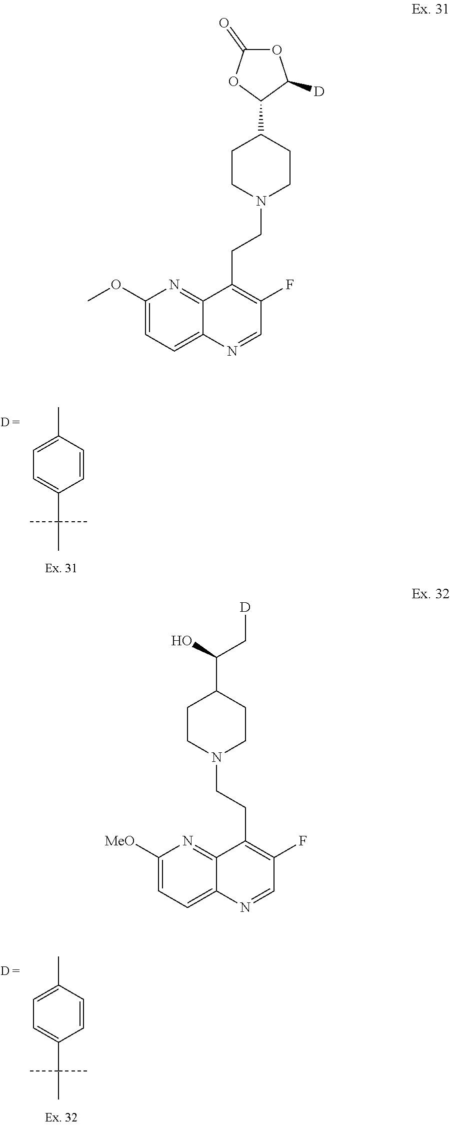

### Method of Use

The compounds disclosed herein can be used to treat infections and inhibit the growth of bacteria. In certain examples, disclosed are methods of treating an infection in a patient, comprising administering to the patient a therapeutically effective amount of any of the compounds disclosed herein. Specific examples of infections that can be treated include, but are not limited to, Actinobacter, Actinomycetes, Bacilli, Bortedellen, Clostridia, Corynebacteria, Enterobacter, Enterococci, Helicobacter, Haemophilus, Klebsiella, Listeria, Mycobacteria, Neisseria, Shigella, Salmonella, Streptococci, Staphylococci, Tuberculosis bacteria, and Yersinia.

In some examples, the disclosed compounds can be used to treat infections caused by resistant G-pos. bacteria such as Methicillin Resistant Staphylococcus aureus (MRSA). Despite newly launched drugs and others in clinical development, the CDC characterizes MRSA as a serious threat, its second highest level of concern. These disclosed methods can involve administering a compound disclosed herein to the infected human or animal or the human or animal at risk of being infected. In some specific examples, the infected individual has cystic fibrosis.

In some examples, the disclosed compounds can be used to treat infections caused by resistant G-neg. pathogens such as P. aeruginosa. Infections caused by G-neg. bacteria in general, and MDR P. aeruginosa in particular (Wagner, S., et al. J. Med. Chem. 2016, 59, 5929), represent a key need in antibacterial drug discovery that is currently underrepresented by approaches in clinical development. The additional permeability barrier imposed by the outer membrane of G-neg. organisms (Zgurskaya, H. I., et al. ACS Infect. Dis. 2015, 1, 512), as well as other resistance mechanisms such as robust multidrug efflux transporters, make the identification of potential new therapies particularly challenging. These disclosed methods can involve administering a compound disclosed herein to the infected human or animal or the human or animal at risk of being infected.

In still other examples, the disclosed compounds can be used to treat infections by M. tuberculosis, M. avium, or M. abscessus.

In some examples, the disclosed compounds can be used to treat infections caused by Enterococcus faecium, Klebsiella pneumoniae, Acinetobacter baumannii, various Enterobacter, and Neisseria gonorrhoeae. Further examples include the following diseases include: tuberculosis; Pneumonia; Typhoid; Paratyphoid; Syphilis, Gastritis; Gastroenteritis; Ruhr; Pestilence; Enteritis; extraintestinal infections, peritonitis and appendicitis with E. coli and intestinal infections with EHEC, EPEC, ETEC and EIEC; Cholera, Legionnaires' disease, whooping cough, brucellosis, Lyme disease, leptospirosis, typhus, trachoma, gonorrhea, meningitis, septicemia, leprosy etc. These methods can involve administering a compound disclosed herein to the infected human or animal or the human or animal at risk of being infected.

In other examples, disclosed herein are methods of treating an infection in a patient, comprising administering to the patient a therapeutically effective amount of any of the compounds disclosed herein.

In these disclosed methods, one can treat humans with infections, but also can treat livestock (horses, cows, pigs, sheep, goats etc.), poultry, and companion animals (dogs, cats, rabbits, etc.). The compositions or organisms can be administered alone or in combination with other therapeutics or nutritional supplements, for example the composition can be combined into a feed.

### Combinations

The disclosed compounds can also be combined with additional antimicrobial agents. For example, the disclosed compounds can be combined with one or more of Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Cefiriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodiumr; Nafcillin Sodium; Nalidixate Sodium; Nalidixic Acid; Natainycin; Nebramycin; Neomycin Palmitate; Neomycin Sulfate; Neomycin Undecylenate; Netilmicin Sulfate; Neutramycin; Nifuiradene; Nifuraldezone; Nifuratel; Nifuratrone; Nifurdazil; Nifurimide; Nifiupirinol; Nifurquinazol; Nifurthiazole; Nitrocycline; Nitrofurantoin; Nitromide; Norfloxacin; Novobiocin Sodium; Ofloxacin; Onnetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisboxazole Diolamine; Sulfomyxin; Sulopenem; Sultamricillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; or Zorbamycin.

The disclosed compounds can also be combined with foaming agents such as sodium laureth ether sulfate (SLES), sodium lauryl dodecyl sulfate (SDS), disodium laureth sulfosuccinate, ammonium lauryl sulfate (ALS), sodium pareth sulfate, and sodium coceth sulfate. Foaming agents can be present at from about 1% to about 70%, about 5% to about 50%, about 10% to about 30%, or about 1% to about 5% by weight.

The disclosed compounds can, in some examples, further comprise one or more antibiotics. Examples of antibiotics include amikacin, gentamicin, kanamycin, neomycin, streptomycin, tobramycin, bacitracin, clindamycin, daptomycin, lincomycin, linezolid, metronidazole, polymyxin, rifaximin, vancomycin, penicillin, cephalosporin, cephazolin, cephalexin, erythromycin, azithromycin, ciprofloxacin, levofloxacin, sulfadiazine, minocycline, tetracycline, and rifampin. The proportion of antibiotics can be about 0.001% to about 10%, about 0.01% to about 5%, about 0.1% to about 10%, or about 1% to about 5% by weight.

The disclosed compounds can, in some examples, further comprise additional agents such as acyclovir, cephradine, malphalen, procaine, ephedrine, adriamycin, dauno, mycin, plumbagin, atropine, quinine, digoxin, and quinidine, cephradine, cephalothin, cishydroxy-L-proline, melphalan, nicotinic acid, nitric oxide, nitroglycerin, chemodeoxycholic acid, chlorambucil, paclitaxel, sirolimus, 5-flurouracil, paclitaxel, mercaptoethanesulfonate, verapamil, or antifungal agents. The proportion of these additional agents can be about 0.001% to about 10%, about 0.01% to about 5%, about 0.1% to about 10%, or about 1% to about 5% by weight.

In some examples, the disclosed compounds can further comprise anti-inflammatory agents. Examples of such agents include acetaminophen, aspirin, celecoxib, diclofenac, diflunisal, flurbiprofen, ibuprofen, indomethacin, ketoprofen, ketorolac, meclofenamate, meloxicam, methyl salicylate, nabumetone, naproxen, oxaprozin, piroxicam, sulindac, tolmetin, trolamine. The proportion of these anti-inflammatory agents can be present in the formulation at from about 1% to about 70%, about 5% to about 50%, about 10% to about 30%, or about 1% to about 5% by weight.

### Administration

The disclosed compounds can be administered either sequentially or simultaneously in separate or combined pharmaceutical formulations. When one or more of the disclosed compounds is used in combination with a second therapeutic agent the dose of each compound can be either the same as or differ from that when the compound is used alone. Appropriate doses will be readily appreciated by those skilled in the art.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrastemal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S.

Application Publication No. 20020035243 also describes compositions for transporting biological moieties across cell membranes for intracellular delivery. Compounds can also be incorporated into polymers, examples of which include poly (D-L lactide-co-glycolide) polymer, poly[bis(p-carboxyphenoxy) propane:sebacic acid] in a 20:80 molar ratio (as used in GLIADEL); chondroitin; chitin; and chitosan.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### General Procedures

Methyl Ketone Arylation: (±)-BINAP (0.036 eq), Pd₂(dba)₃ (0.015 eq) and Na^{t}BuO (1.3 eq) were added to round bottom flask and purged with nitrogen. THF (0.17 M) was added to the round bottom followed by the bromide (1 eq), and methyl ketone (1.2 eq). This stirred at 70° C. overnight. Upon return, the reaction was cooled to room temperature and diluted with water. This was then extracted from 5×15 mL ethyl acetate. Ethyl acetate was then dried with sodium sulfate, decanted, and concentrated to afford crude mixtures. Crude mixtures were purified by flash chromatography.

Deprotection of Boc Group: Boc-protected compound (1 eq) was dissolved in methanol (0.25 M). Next, 4 M HCl in 1,4-dioxane (2-2.5 eq) was added dropwise. This stirred for 4 hours. The stir bar was removed and the reaction was concentrated to afford salts. No purification was done at this step.

Reductive Amination on Aldehyde: Aldehyde (1.0 eq), piperidine (1.1 eq) and 4A molecular sieves (350 mg/mmol of aldehyde) were added to round bottom flask. Methanol and THF (1:1, 0.25 M) were added to the reaction, followed by acetic acid (1.5 eq). This stirred for 4 hours. Then sodium cyanoborohydride (1.2 or 2.0 eq) was added and the reaction stirred for one more hour. The reaction was diluted with aqueous sodium carbonate, and the aqueous layer was extracted 5×15 mL ethyl acetate. Organics were collected, dried with sodium sulfate, decanted, and concentrated to afford crude mixtures. These mixtures were purified by flash chromatography.

Ketone Reduction: Starting material (1.0 eq) was dissolved in methanol (0.2 M). Sodium borohydride (1.2 eq) was added to reaction and was allowed to stir, monitoring by TLC for consumption of starting material. Once starting material was consumed, the reaction was diluted with aqueous sodium carbonate and extracted 5×10 mL 10% methanol in dichloromethane. Then organics were collected, dried over sodium sulfate, decanted, and concentrated to afford crude mixtures. Crude mixtures were then purified by flash chromatography.

Reductive Amination on Ketone: Ketone (1.0 eq) and racemic tert-butyl sulfinamide (1.2 eq) were added to round bottom flask. The round bottom flask was sparged with nitrogen followed by the addition of THF (0.2 M) and titanium ethoxide (1 eq). This stirred at 70° C. overnight. Upon return, the reaction is cooled to room temperature and methanol (equivalent amount to THF) added to the reaction. Then sodium borohydride (1.2 eq) was added. This stirred an additional 30 minutes before reaction was diluted with aqueous sodium carbonate. This was then extracted 5×15 mL ethyl acetate. Organics were collected, dried over sodium sulfate, decanted, and concentrated by rotary evaporation to afford crude mixtures. Crude mixtures were then used directly in the next step without purification.

Deprotection of Sulfinamide: Starting material (1 eq) was dissolved in methanol (0.2 M). 4 M HCl (2 eq) was added to reaction and allowed to stir, monitoring by TLC. Once starting material was consumed, the reaction was basified to pH of ˜12 with sodium hydroxide. This mixture was extracted 5×10 mL 10% methanol in dichloromethane. Then organics were collected, dried over sodium sulfate, decanted, and concentrated by rotary evaporation to afford crude mixtures. Crude mixtures were purified by flash chromatography.

Photo-Heck Coupling: 4-CZIPN (0.05 eq), Co(dmgH)(dmgH₂)Cl₂ (0.03 eq), tert-butyl 4-iodopiperidine-1-carboxylate (1.0 or 2.0 eq), dibasic potassium phosphate (2.0 eq), and alkene starting material (1.0 eq) were added to round bottom flask. Then dimethyl formamide (0.1 M) was added to round bottom, followed by triethylamine (2 eq). The reaction was irradiated with 440 nm blue light, positioned 5-20 cm from the flask, for 17-24 hours, monitoring by TLC. This stirred for 17-24 hours, monitoring by TLC. Once alkene starting material was consumed, the reaction was stopped by turning off the light. The reaction was diluted with water and extracted 5×15 mL ethyl acetate. Organics were collected, dried over sodium sulfate, decanted, and concentrated by rotary evaporation to afford crude mixtures. Crude mixtures were separated by flash chromatography.

Alkene Hydrogenation: Alkene starting material (1 eq) was dissolved in methanol (0.2 M). 10% Pd/C (0.1 eq) was added to the reaction. The reaction was sparged with 1× nitrogen balloon, followed by 1× hydrogen balloon. Then a hydrogen balloon was attached to the reaction flask by needle and allowed to stir overnight. Upon return, reaction was sparged with 1× nitrogen balloon, and filtered through celite plug washing with methanol. Organics were concentrated by rotary evaporation to afford crude mixtures. Crude mixtures were purified by flash chromatography.

Asymmetric Dihydroxylation: Alkene starting material (1 eq) was dissolved in tert-butanol (0.1 M), diluted with water (0.1 M), and respective AD-mix (1.2 g/mmol starting material) was added. This stirred overnight, monitoring by TLC. Once starting material was consumed, the reaction was quenched with sodium sulfite and allowed to stir for 15 more minutes. The reaction was diluted with water and extracted 5×10 mL 10% methanol in dichloromethane. Then organics were collected, dried over sodium sulfate, decanted, and concentrated by rotary evaporation to afford crude mixtures. Crude mixtures were purified by flash chromatography.

Wittig Reaction: ((1-(tert-butoxycarbonyl)piperidin-4-yl)methyl)triphenylphosphonium iodide (2.0 eq) was suspended in THF (0.2 M). Potassium tert-butoxide (1 M solution in THF, 2.0 eq) was added to the reaction. This stirred for 1 hour, becoming orange in color. Then the reaction was cooled to 0° C. and stirred an additional 5 mins. Aldehyde (1.0 eq) was added to the reaction and the reaction stirred overnight, warming to room temperature. Upon return, the reaction was quenched with aqueous ammonium chloride. This mixture was filtered through a celite plug, washing with 3 volumes of ethyl acetate. Organics were dried over sodium sulfate, decanted, and concentrated by rotary evaporation to afford crude mixtures. Crude mixtures were purified by flash chromatography.

Photoisomerization: Alkene starting material (1.0 eq) was added to round bottom flask and dissolved in acetonitrile (0.2 M). [4,4′-Bis(1,1-dimethylethyl)-2,2′-bipyridine-N1,N1′]bis[3,5-difluoro-2-[5-(trifluoromethyl)-2-pyridinyl-N]phenyl-C] Iridium(III) hexafluorophosphate was added (0.01 eq). Then the reaction was irradiated with 456 nm blue light, positioned 5-20 cm from the flask, overnight. Upon return, the reaction was stopped. Solvent was removed by rotary evaporation to afford crude mixtures. Mixtures were then purified by flash chromatography.

Bromohydrin Synthesis: Alkene starting material (1 eq) was added to round bottom flask and dissolved in acetonitrile (0.2 M). Water (0.2 M [1:1 ratio with acetonitrile]) was added to the round bottom. The solution was sparged with 1× nitrogen balloon. Reaction was cooled to 0° C. and N-bromosuccinimide (1.2 eq) was added. Reaction was then covered in foil and allowed to stir overnight. Upon return, the reaction was quenched with aqueous sodium thiosulfate. Reaction became yellow upon addition of sodium thiosulfate. Reaction was diluted with ethyl acetate and organics were separated from aqueous layer. The aqueous layer was extracted 5×5 mL ethyl acetate. Organics were combined, dried over sodium sulfate, decanted, and concentrated by rotary evaporation to afford crude mixtures. Crude mixtures were then purified via flash chromatography.

Epoxide Formation: Bromohydrin (1 eq) was dissolved in methanol (0.2 M). Potassium carbonate (2 eq) was added and allowed to stir until consumption of starting material was observed by TLC. Upon completion of reaction, the reaction was quenched with aqueous ammonium chloride and filtered through celite plug, washing with ethyl acetate. Then aqueous and organic layers were separated. Organics were dried over sodium sulfate, decanted, and concentrated by rotary evaporation to afford desired epoxide. No purification was done at this stage.

Hydrogenation of Epoxide: Epoxide (1 eq) was dissolved in ethanol (0.2 M). 10% Pd/C (5 mg) was added to the reaction. 1× nitrogen balloon was used to sparge the reaction solution followed by 1× hydrogen balloon. Finally, 1× hydrogen balloon was attached to reaction by needle and allowed to stir overnight. Upon return, the reaction was sparged with 1× nitrogen balloon, filtered through celite plug, and washed with ethyl acetate. Then solvent was removed by rotary evaporation to afford crude mixtures. No purification was done at this stage.

Hydroxyl Oxidation to Ketone: Alcohol starting material (1 eq) was dissolved in DCM (0.2 M) and cooled to 0° C. Dess Martin-Periodinane (1.2 eq) was added in one portion. Reaction began bubbling upon addition. This was allowed to stir overnight. Upon return, the reaction was stopped and diluted with water and 1M NaOH. Next, organics were separated from aqueous layer, and aqueous layer was extracted 5×10 mL ethyl acetate. Organics were collected, dried over sodium sulfate, decanted, and concentrated by rotary evaporation to afford desired compound.

Carbonate Synthesis: 1-(1H-imidazole-1-carbonyl)-1H-imidazole (1.5 eq) and diol starting material (1 eq) were added to round bottom flask. This was dissolved in 2-butanone (0.057 M) and triethyl amine (2 eq) was added to reaction. This was allowed to stir for 48 hours at 60° C. Upon return, the reaction was cooled to room temperature and diluted with water and ethyl acetate. Organics were separated and the aqueous layer was extracted 3×15 mL ethyl acetate. Organics were collected, dried over sodium sulfate, decanted, and concentrated by rotary evaporation to afford crude mixtures. Mixtures were purified via flash chromatography.

Deoxygenative Arylation: Diol-starting material (0.2 mmol) and pyridinium triflate (0.2 mmol) were added to round bottom flask, along with a magnetic stir bar, and were nitrogen purged. Then 2 mL 1,4-dioxane was added and all material dissolved. Carbene precursor 20 (0.22 mmol) was added in 2 portions, with 10 mins stirring between each addition. After addition, this was allowed to stir while the reaction vessel was prepped. To the reaction vessel, the requisite (hetero)aryl bromide (0.3 mmol), nickel catalyst (0.02 mmol), iridium catalyst (0.005 mmol), quinuclidine (0.6 mmol), and dimethylacetamide (DMA, 2 mL) were added. This stirred for 5 min, then the alcohol solution was added to the reaction vessel. This was degassed with nitrogen for 10 mins, then allowed to stir in front of a 456 nm blue light for 3 hours. The reaction was then diluted with 20 mL water and extracted 5×10 mL of 10% methanol in dichloromethane. The combined organic layers were dried over anhydrous sodium sulfate, decanted, concentrated by rotary evaporation, and purified by flash chromatography on silica gel.

General UPLC Conditions: Measurements of purity were carried out using a Acquity UPLC BEH C₁₈ column (2.1×50 mm i.d., 1.7 μm). The separations were carried out at 40° C. at a flow rate of 0.7 mL/min in gradient mode of mobile phases of water+0.1% formic acid and acetonitrile+0.1% formic acid. The concentration of tested compounds was ˜0.5 mg/mL with 2 μl injection volume. Peak detection was accomplished using a UV detector scanning from 210-500 nm.

General Chiral HPLC Conditions: Measurements of enantiomeric excess (ee) were carried out using a CHIRALPAK™ IB N-3 column (150*4.6 mm i.d., 3 μm). The separations were carried out at 25° C. at a flow rate of 1 mL/min in isocratic mode of mobile phases containing hexane and reagent alcohol, with diethylamine as an additive. The concentration of tested compounds was ˜250 μg/ml in ethanol with 30 μL injection volume. Peak detection was accomplished using a UV detector at 220 nm.

### Characterization

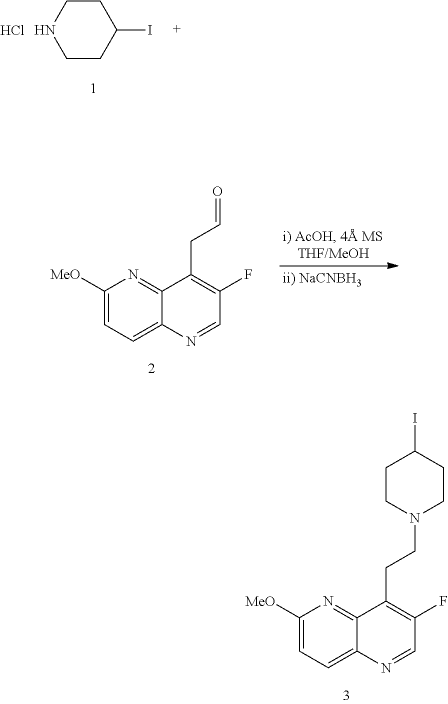 [SMILES: Cl ; IC1CCNCC1 ; COc1ccc2ncc(F)c(CC=O)c2n1 ; COc1ccc2ncc(F)c(CCN3CCC(I)CC3)c2n1]

7-fluoro(2-(4-iodopiperidin-1-ethyl)-2-methoxy-1,5-naphthyridine (3): Title compound was synthesized from piperidine 1 and aldehyde 2 using General Procedure 3. Purification by flash chromatography (compound eluted in 15% ethyl acetate in hexanes) and concentration in vacuo afforded the title compound as a yellow solid (265 mg, 0.6396 mmol, 64% yield).

¹HNMR (CDCl₃, 400 MHz) δ: 8.62 (s, 1H), 8.18 (d, J=9.0 Hz, 1H), 7.08 (d, J=9.1 Hz, 1H), 4.34 (br s, 1H), 4.08 (s, 3H), 3.50-3.32 (br s, 2H), 2.94-2.66 (m, 4H), 2.57-2.30 (m, 2H), 2.25-2.09 (m, 4H).

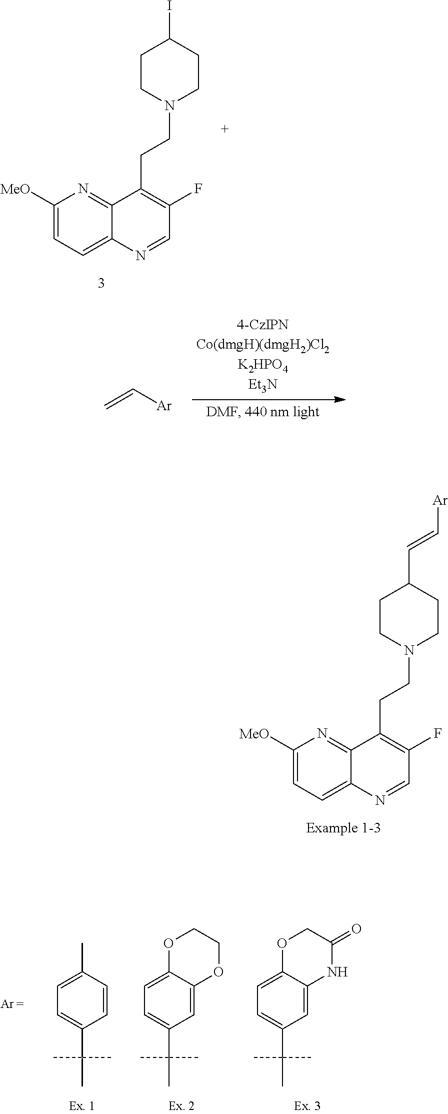

Example 1. (E)-7-fluoro-2-methoxy-8-(2-(4-(4-methylstyryl)piperidin-1-yl)ethyl)-1,5-naphthyridine: Title compound was synthesized from iodide 3 and 1-methyl-4-vinylbenzene using General Procedure 7. Purification by flash chromatography (compound eluted in 40% ethyl acetate in hexanes) and concentration in vacuo afforded the title compound as a yellow solid (158 mg, 0.390 mmol, 40% yield).

¹HNMR (CD₃OD, 400 MHz) δ: 8.64 (s, 1H), 8.21 (d, J=9.1 Hz, 1H), 7.23 (d, J=8.0 Hz, 2H), 7.18 (d, J=9.1 Hz, 1H), 7.08 (d, J=8.0 Hz, 2H), 6.38 (d, J=16.0 Hz, 1H), 6.15 (dd, J=16.0, 6.9 Hz, 1H), 4.12 (s, 3H), 3.53-3.47 (m, 2H), 3.22-3.15 (m, 2H), 2.84-2.77 (m, 2H), 2.32-2.14 (m, 3H), 2.29 (s, 3H), 1.88-1.79 (m, 2H), 1.63-1.51 (m, 2H). ¹³CNMR (CDCl₃, 100 MHz) δ: 162.55, 157.43 (d, J=255.2 Hz), 141.80 (d, J=7.1 Hz), 140.29, 138.66 (d, J=2.0 Hz), 138.06 (d, J=28.1 Hz), 136.83, 135.05, 134.21, 130.72 (d, J=12.6 Hz), 129.33, 128.16, 126.03, 115.33 (d, J=2.6 Hz), 57.59, 53.97, 53.52, 39.47, 32.34, 21.27, 21.06. HRMS (ESI) m/z calc'd for C₂₅H₂₉FN₃O [M+H]⁺: 406.22892; found: 406.2283. UPLC: rt: 3.93 min, purity: 95.7%.
Example 2. (E)-8-(2-(4-(2-(2,3-dihydrobenzo[b][1,4]dioxin-6-yl)vinyl)piperidin-1-yl)ethyl)-7-fluoro-2-methoxy-1,5-naphthyridine: Title compound was synthesized from iodide 3 and 6-vinyl-2,3-dihydrobenzo[b][1,4]dioxine using General Procedure 7. Purification by flash chromatography (compound eluted in 20% ethyl acetate in hexanes) and concentration in vacuo afforded the title compound as a white solid (105 mg, 0.234 mmol, 28% yield).

¹HNMR (DMSO-d₆, 400 MHz) δ: 8.77 (s, 1H), 8.28 (d, J=9.0 Hz, 1H), 7.24 (d, J=9.0 Hz, 1H), 6.86 (d, J=1.9 Hz, 1H), 6.84 (dd, J=2.0, 8.3 Hz, 1H), 6.75 (d, J=8.2 Hz, 1H), 6.23 (d, J=16.0 Hz, 1H), 6.06 (dd J=6.8, 16.1 Hz, 1H), 4.21 (s, 4H), 4.05 (s, 3H), 3.33-3.29 (m, 2H, obscured by water) 3.01-2.98 (m, 2H), 2.69-2.66 (m, 2H), 2.10-2.02 (m, 3H), 1.68-1.65 (m, 2H), 1.32 (qd, J=3.6, 12.4 Hz, 2H). ³CNMR (DMSO-d₆, 100 MHz) δ: 161.96, 156.82 (d, J=254.1 Hz), 143.29, 142.48, 140.92 (d, J=7.1 Hz), 140.40, 138.07 (d, J=2.3 Hz), 137.86 (d, J=27.8 Hz), 133.36, 130.86, 126.90, 118.92, 116.96, 115.28 (d, J=2.5 Hz), 114.18, 64.01 (d, J=5.5 Hz), 56.86, 53.54, 52.75, 38.61, 31.81, 20.66. HRMS (ESI) m/z calc'd for C₂₆H₂FN₃O₃ [M+H]⁺: 450.21875; found: 450.21790. UPLC: rt: 3.693 min, purity: 94.1%.
Example 3. (E)-6-(2-(1-(2-(3-fluoro-6-methoxy-1,5-naphthyridin-4-yl)ethyl)piperidin-4-yl)vinyl)-2H-benzo[b][1,4]oxazin-3(4H)-one: Title compound was synthesized from iodide 3 and 6-vinyl-2H-benzo[b][1,4]oxazin-3(4H)-one using General Procedure 7. Purification by flash chromatography (compound eluted in 20% ethyl acetate in hexanes) and concentration in vacuo afforded the title compound as a yellow solid (66 mg, 0.143 mmol, 31% yield).

¹HNMR (CDCl₃, 400 MHz) δ: 8.62 (s, 1H), 8.18 (d, J=9.0 Hz, 1H), 7.84 (s, 1H), 7.07 (d, J=9.0 Hz, 1H), 6.96 (dd, J=1.8, 8.3 Hz, 1H), 6.90 (d, J=8.3 H, 1H), 6.77 (d, J=1.8 Hz, 1H), 6.28 (d, J=15.9 Hz, 1H), 6.05 (dd, J=7.0, 15.9 Hz, 1H), 4.60 (s, 2H), 4.09 (s, 3H), 3.44-3.40 (m, 2H), 3.13-3.11 (m, 2H), 2.77-2.74 (m, 2H), 2.19 (td, J=2.1, 15.9 Hz, 2H), 2.18-2.10 (m, 1H), 1.81-1.75 (m, 2H), 1.60-1.50 (obscured by water, m, 2H). ¹³CNMR (CDCl₃, 100 MHz) δ: 165.45, 162.55, 142.88, 141.82 (d, J=7.3 Hz), 140.32, 138.68, 138.08, 137.93, 135.05, 133.11, 126.59 (d, J=74.6 Hz), 122.24, 117.06, 115.34 (d, J=2.8 Hz), 113.06, 67.56, 57.61, 53.95, 53.50, 39.46, 32.34, 21.13. HRMS (ESI) m/z calc'd for C₆H₂FN₄O₃ [M+H]⁺: 463.21400; found: 463.21280. UPLC: rt: 3.37 min, purity: 96.8%.

The absolute stereochemistry in Examples 4-6 and 7-9 was assigned on the basis of the established stereoselectivity of the Sharpless asymmetric dihydroxylation (Sharpless, K. B., et al. J. Org. Chem. 1992, 57, 2768-2771).

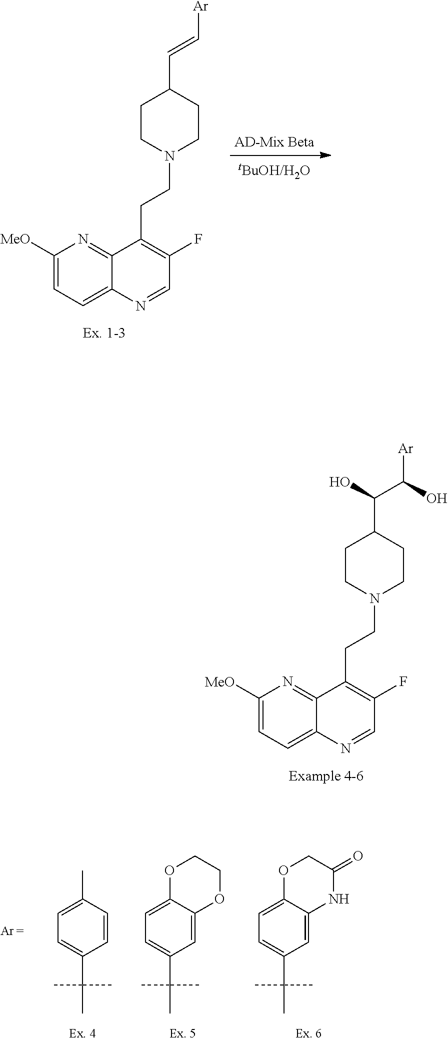

Example 4. (1R,2R)-1-(1-(2-(3-fluoro-6-methoxy-1,5-naphthyridin-4-yl)ethyl)piperidin-4-yl)-2-(p-tolyl)ethane-1,2-diol: Title compound was synthesized from Example 1 using General Procedure 9 with AD-mix β. Purification by flash chromatography (compound eluted in 100% DCM) and concentration in vacuo afforded the title compound as a clear oil (55 mg, 0.148 mmol, 60% yield).

¹HNMR (CD₃OD, 400 MHz) δ: 8.63 (s, 1H), 8.20 (d, J=9.1 Hz, 1H), 7.25 (d, J=8.0 Hz, 2H), 7.19-7.13 (m, 3H) 4.59 (d, J=5.8 Hz. 1H), 4.10 (s, 3H), 3.50-3.40 (m, 3H), 3.23-3.14 (m, 2H), 2.83-2.73 (m, 2H), 2.32 (s, 3H), 2.19-2.04 (m, 2H), 1.91-1.83 (m, 1H), 1.70-1.49 (m, 3H), 1.42-1.32 (m, 1H). ¹³CNMR (DMSO-d₆, 100 MHz, contaminated w/DMSO) δ: 161.97, 156.80 (d, J=254.1 Hz), 140.90, 140.83 (d, J=1.3 Hz), 140.68, 140.38, 138.05 (d, J=2.2 Hz), 137.84 (d, J=28.0 Hz), 135.57, 128.28, 126.62, 115.27, 78.01, 73.18, 56.82. 53.53, 53.09, 37.49, 29.00, 26.43, 20.65. HRMS (ESI) m/z calc'd for C₂₅H₃₁FN₃O₃ [M+H]⁺: 440.2344; found: 440.23349. UPLC: rt: 3.27 min, purity: 93.3%. Enantiomeric excess (ee) undetermined.
Example 5. (1R,2R)-1-(2,3-dihydrobenzo[b][1,4]dioxin-6-yl)-2-(1-(2-(3-fluoro-6-methoxy-1,5-naphthyridin-4-yl)ethyl)piperidin-4-yl)ethane-1,2-diol: Title compound was synthesized from Example 2 using General Procedure 9 with AD-mix β. Purification by flash chromatography (compound eluted in 100% ethyl acetate) and concentration in vacuo afforded the title compound as a clear oil (16 mg, 0.033 mmol, 20% yield).

¹HNMR (DMSO-d₆, 400 MHz, contaminated with ethyl acetate) δ: 8.76 (s, 1H), 8.27 (d, J=9.0 Hz, 1H), 7.23 (d, J=9.0 Hz, 1H), 6.80 (s, 1H), 6.77-6.72 (m, 2H), 4.92 (d, J=5.0 Hz, 1H), 4.36 (t, J=5.2 Hz, 1H), 4.25 (d, J=5.9 Hz, 1H), 4.20 (s, 4H), 4.03 (s, 3H), 3.30-3.24 (m, 2H), 3.14 (q, J=5.4 Hz, 1H), 3.01-2.92 (m, 2H), 2.61 (t, J=7.4 Hz, 2H), 1.92-1.78 (m, 2H), 1.71-1.63 (m, 1H), 1.47-1.20 (m, 4H). ¹³CNMR (DMSO-d₆, 100 MHz, contaminated w/ ethyl acetate) δ: 161.96, 156.83 (d, J=254.2 Hz), 142.75, 142.07, 140.93 (d, J=7.0 Hz), 140.41, 138.08 (d, J=2.1 Hz), 137.86 (d, J=27.8 Hz), 136.98, 119.46, 116.18, 115.39, 115.27 (d, J=2.2 Hz), 78.00, 72.75, 63.99 (d, J=3.5), 56.89, 53.54, 53.20, 37.61. 29.05, 26.60, 20.68. HRMS (ESI) m/z calc'd for C₆H₃₁FN₃O₅ [M+H]⁺: 484.22423; found: 484.2233. UPLC: rt: 3.073 min, purity: 94.2%. Enantiomeric excess (ee) determined to be 99% by chiral HPLC (90/10 hexane/reagent alcohol with 0.1% diethylamine as an additive).
Example 6. 6-((1R,2R)-2-(1-(2-(3-fluoro-6-methoxy-1,5-naphthyridin-4-yl)ethyl)piperidin-4-yl)-1,2-dihydroxyethyl)-2H-benzo[b][1,4]oxazin-3(4H)-one: Title compound was synthesized from Example 3 using General Procedure 9 with AD-mix β. Purification by flash chromatography (compound eluted in 1% methanol in DCM) and concentration in vacuo afforded the title compound as a white solid (12 mg, 0.024 mmol, 22% yield).

¹HNMR (DMSO-d₆, 400 MHz) δ: 10.63 (s, 1H), 8.77 (s, 1H), 8.28 (d, J=9.0 Hz, 1H). 7.24 (d, J=9.0 Hz, 1H), 6.90 (m, 1H), 6.85 (m, 2H), 5.02 (d, J=4.8 Hz, 1H), 4.53 (s, 2H), 4.41 (t. J=4.8 Hz, 1H), 4.31-4.30 (m, 1H), 4.03 (s, 3H), 3.33-3.26 (m, 2H, obscured by water), 3.15-3.11 (m, 1H), 2.98-2.95 (m, 2H), 2.63-2.59 (m, 2H), 1.90-1.82 (m, 2H), 1.68-1.65 (m, 1H), 1.46-1.34 (m, 2H), 1.27-1.19 (m, 2H). ¹³CNMR (DMSO-d₆, 100 MHz) δ: 164.93, 161.96, 156.82 (d, J=254.1 Hz), 141.98, 140.91 (d, J=7.1 Hz), 140.40, 138.20, 138.08 (d, J=1.5 Hz), 137.86 (d, J=27.8 Hz), 126.65, 121.30, 115.28, 114.28, 78.04, 72.71, 66.76, 56.80, 53.54, 53.12, 37.51, 29.00, 26.68, 20.61. HRMS (ESI) m/z calc'd for C₆H₃₀FN₄O₅ [M+H]⁺: 497.21947; found: 497.21884. UPLC: rt: 2.917 min, purity: 95.6%. Enantiomeric excess (ee) determined to be 99% by chiral HPLC (90/10 hexane/reagent alcohol with 0.1% diethylamine as an additive).

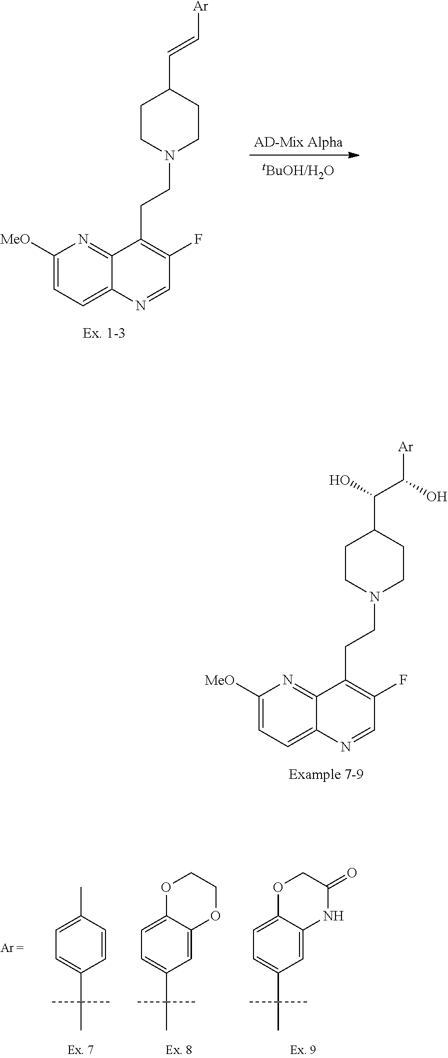

Example 7. (1S,2S)-1-(1-(2-(3-fluoro-6-methoxy-1,5-naphthyridin-4-yl)ethyl)piperidin-4-yl)-2-(p-tolyl)ethane-1,2-diol: Title compound was synthesized from Example 1 using General Procedure 9 with AD-mix α. Purification by flash chromatography (compound eluted in 100% ethyl acetate) and concentration in vacuo afforded the title compound as a clear oil (65 mg, 0.148 mmol, 60% yield).

¹HNMR (CD₃OD, 400 MHz) δ: 8.63 (s, 1H), 8.20 (d, J=9.1 Hz, 1H), 7.25 (d, J=8.0, 2H), 7.19-7.13 (m, 3H), 4.59 (d, J=5.7 Hz, 1H), 4.10 (s, 3H), 3.50-3.40 (m, 3H), 3.21-3.11 (m, 2H), 2.79-2.71 (m, 2H), 2.32 (s, 3H), 2.14-2.02 (m, 2H), 1.90-1.82 (m, 1H), 1.68-1.47 (m, 3H), 1.41-1.31 (m, 1H). ¹³CNMR (DMSO-d₆, 100 MHz, contaminated w/ethyl acetate) δ: 161.95, 156.82 (d, J=254.1 Hz), 140.91 (d, J=7.1 Hz), 140.75, 140.39, 138.07 (d, J=2.2 Hz), 137.84 (d, J=27.8 Hz), 135.59, 128.30, 126.64, 115.25 (d, J=2.0 Hz), 78.09, 73.20, 56.86, 53.52, 53.18, 37.57, 29.09, 26.49, 20.67. HRMS (ESI) m/z calc'd for C₂₅H₃₁FN₃O₃ [M+H]⁺: 440.2344; found: 440.23407. UPLC: rt: 3.271 min, purity: 95.6%. Enantiomeric excess (ee) undetermined.
Example 8. (1S,2S)-1-(2,3-dihydrobenzo[b][1,4]dioxin-6-yl)-2-(1-(2-(3-fluoro-6-methoxy-1,5-naphthyridin-4-yl)ethyl)piperidin-4-yl)ethane-1,2-diol: Title compound was synthesized from Example 2 using General Procedure 9 with AD-mix α. Purification by flash chromatography (compound eluted in 1% methanol in DCM) and concentration in vacuo afforded the title compound as a clear oil (15 mg, 0.031 mmol, 12% yield).

¹HNMR (DMSO-d₆, 400 MHz, contaminated with DMSO) δ: 8.76 (s, 1H), 8.27 (d, J=9.0 Hz, 1H), 7.23 (d, J=9.0 Hz, 1H), 6.80 (s, 1H), 6.77-6.72 (m, 2H), 4.92 (d, J=4.9 Hz, 1H), 4.36 (t, J=5.2 Hz, 1H), 4.26 (br d, J=5.5 Hz, 1H), 4.2 (s, 4H), 4.03 (s, 3H), 3.31-3.23 (m, 2H, partially obscured by water), 3.18-3.12 (m, 1H, partially obscured by methanol), 3.03-2.92 (m, 2H), 2.69-2.57 (m, 2H), 1.96-1.77 (m, 2H), 1.72-1.63 (m, 1H), 1.49-1.11 (m, 4H). ¹³CNMR (DMSO-d₆, 100 MHz) δ: 161.98, 156.84 (d, J=254.2 Hz), 142.77, 142.08, 140.93 (d, J=7.1 Hz), 140.43, 138.09 (d, J=2.2 Hz), 137.88 (d, J=27.7 Hz), 136.98, 119.48, 116.20, 115.41, 115.30 (d, J=2.1 Hz), 77.98, 72.77, 64.00 (d, J=3.6 Hz), 56.84, 53.57. 53.16, 37.55, 29.00, 26.54, 20.65. HRMS (ESI) m/z calc'd for C₂₆H₃₁FN₃O₅ [M+H]⁺: 484.22423; found: 484.22348. UPLC: rt: 3.103 min, purity: 97.6%. Enantiomeric excess (ee) determined to be 97.8% by chiral HPLC (90/10 hexane/reagent alcohol with 0.1% diethylamine as an additive).
Example 9. 6-((1S,2S)-2-(1-(2-(3-fluoro-6-methoxy-1,5-naphthyridin-4-yl)ethyl)piperidin-4-yl)-1,2-dihydroxyethyl)-2H-benzo[b][1,4]oxazin-3(4H)-one: Title compound was synthesized from Example 3 using General Procedure 9 with AD-mix α. Purification by flash chromatography (compound eluted in 5% methanol in DCM) and concentration in vacuo afforded the title compound as a white solid (11 mg, 0.022 mmol, 20% yield).

¹HNMR (DMSO-d₆, 400 MHz) δ: 10.64 (s, 1H), 8.77 (s, 1H), 8.27 (d, J=9.0 Hz, 1H), 7.23 (d, J=9.0 Hz, 1H), 6.90 (m, 1H), 6.85 (m, 2H), 5.04 (d, J=4.1 Hz, 1H), 4.52 (s, 2H), 4.41 (t. J=4.9 Hz, 1H), 4.35-4.30 (m, 1H), 4.03 (s, 3H), 3.33-3.27 (m, 2H, obscured by water), 3.14 (q, J=5.0 Hz, 1H), 3.03-2.95 (m, 2H), 2.68-2.60 (m, 2H), 1.95-1.83 (m, 2H), 1.69-1.67 (m, 1H), 1.49-1.43 (m, 1H), 1.42-1.31 (m, 2H), 1.28-1.17 (m, 2H). ¹³CNMR (DMSO-d₆, 100 MHz) δ: 164.94, 162.00, 156.82 (d, J=254.2 Hz), 141.99, 140.90 (d, J=6.9 Hz), 140.42, 138.17, 138.09 (d, J=1.4 Hz), 137.74, 126.66, 121.32, 115.30, 114.29, 77.97, 72.73, 66.77, 56.62, 53.59, 53.03, 37.37, 28.83, 26.50, 20.47. HRMS (ESI) m/z calc'd for C₂₆H₃₀FN₄O₅ [M+H]⁺: 497.21947; found: 497.2186. UPLC: rt: 2.915 min, purity: 95.8%. Enantiomeric excess (ee) determined to be 99% by chiral HPLC (90/10 hexane/reagent alcohol with 0.1% diethylamine as an additive).

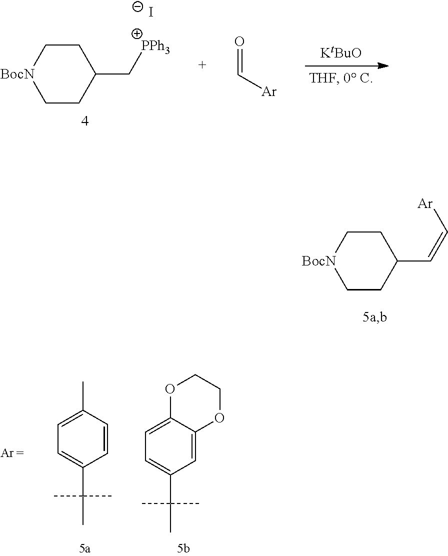

tert-butyl (Z)-4-(4-methylstyryl)piperidine-1-carboxylate (5a): Title compound was synthesized from phosphonium salt 4 and 4-methylbenzaldehyde using General Procedure 10. Purification by flash chromatography (compound eluted in 2% ethyl acetate in hexanes) and concentration in vacuo afforded the title compound as a clear oil (440 mg, 1.46 mmol, 73% yield).

¹HNMR (CDCl₃, 400 MHz) δ: 7.14 (s, 4H), 6.36 (d, J=11.7 Hz, 1H), 5.41 (dd, J=10, 11.6 Hz, 1H), 4.10-4.05 (m, 2H), 2.77-2.66 (m, 3H), 2.35 (s, 3H), 1.70-1.64 (m, 2H), 1.46 (s, 9H), 1.41-1.30 (m, 2H).

tert-butyl (Z)-4-(2-(2,3-dihydrobenzo[b][1,4]dioxin-6-yl)vinyl)piperidine-1-carboxylate (5b): Title compound was synthesized from phosphonium salt 4 and 2,3-dihydrobenzo[b][1,4]dioxine-6-carbaldehyde using General Procedure 10. Purification by flash chromatography (compound eluted in 5% ethyl acetate in hexanes) and concentration in vacuo afforded the title compound as a yellow oil (65% average yield).

¹HNMR (CDCl₃, 400 MHz) δ: 6.83 (d, J=8.3 Hz, 1H), 6.77 (d, J=2.0 Hz, 1H), 6.73 (dd, J=2.0, 8.2 Hz, 1H), 6.27 (d, J=11.6 Hz, 1H), 5.36 (dd, J=10.0, 11.6 Hz, 1H), 4.27 (s, 4H), 4.13-4.01 (m, 2H), 2.80-2.67 (m, 3H), 1.70-1.63 (m, 2H), 1.46 (s, 9H), 1.40-1.28 (m, 2H).

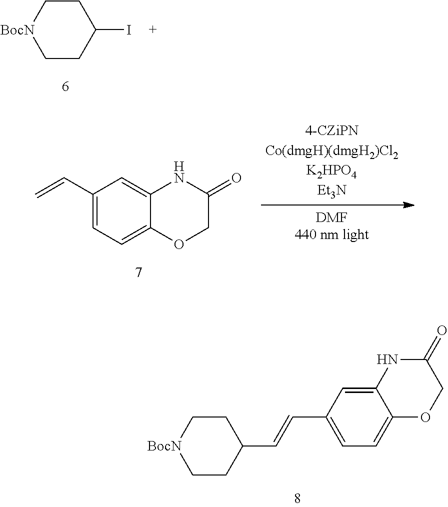 [SMILES: CC(C)(C)OC(=O)N1CCC(I)CC1 ; C=Cc1ccc2c(c1)NC(=O)CO2 ; CC(C)(C)OC(=O)N1CCC(/C=C/c2ccc3c(c2)NC(=O)CO3)CC1]

tert-butyl (E)-4-(2-(3-oxo-3,4-dihydro-2H-benzo[b][1,4]oxazin-6-yl)vinyl)piperidine-1-carboxylate (8): Title compound was synthesized from iodide 6 and 6-vinyl-3,4-dihydro-2H-benzo[b][1,4]oxazine 7 using General Procedure 7. Purification by flash chromatography (compound eluted in 10% ethyl acetate in hexanes) and concentration in vacuo afforded the title compound as a yellow oil (750 mg, 2.09 mmol, 39% yield).

¹HNMR (CDCl₃, 400 MHz, contaminated w/ DCM and ethyl acetate) δ: 7.44 (s, 1H), 6.96 (dd, J=1.8, 8.4 Hz, 1H), 6.91 (d, J=8.3 Hz, 1H), 6.75 (d. J=1.9 Hz. 1H), 6.28 (d, J=16 Hz, 1H), 6.02 (dd, J=6.9, 16 Hz, 1H), 4.60 (s, 2H), 4.18-4.06 (m, 2H), 2.82-2.73 (m, 2H), 2.32-2.22 (m, 1H), 1.77-1.70 (m, 2H), 1.47 (s, 9H), 1.42-1.31 (m, 2H).

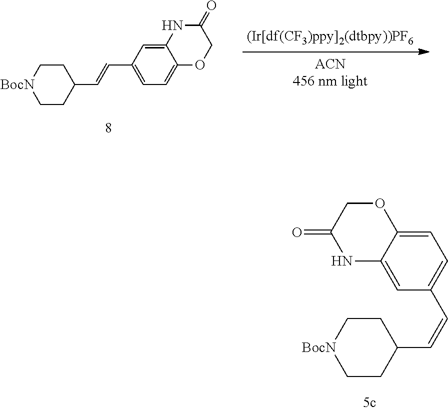 [SMILES: CC(C)(C)OC(=O)N1CCC(/C=C/c2ccc3c(c2)NC(=O)CO3)CC1 ; CC(C)(C)OC(=O)N1CCC(/C=C\c2ccc3c(c2)NC(=O)CO3)CC1]

tert-butyl (Z)-4-(2-(3-oxo-3,4-dihydro-2H-benzo[b][1,4]oxazin-6-yl)vinyl)piperidine-1-carboxylate (5c): Title compound was synthesized from (E)-olefin 8 using General Procedure 11. Purification by flash chromatography (compound eluted in 15% ethyl acetate in hexanes) and concentration in vacuo afforded the title compound as a yellow oil (340 mg, 0.949 mmol, 34% yield). ¹HNMR (CDCl₃, 400 MHz, contaminated w/ DCM) δ: 8.09 (s, 1H), 6.95 (d, J=8.3 Hz, 1H), 6.86 (dd. J=1.8, 8.4 Hz, 1H), 6.65 (d, J=1.7 Hz, 1H), 6.29 (d, J=11.6 Hz, 1H), 5.43 (dd, J=10.0, 11.5 Hz, 1H), 4.62 (s, 2H), 4.15-4.02 (m, 2H), 2.74 (t, J=12.5 Hz, 2H), 2.70-2.59 (m, 1H). 1.69-1.61 (m, 2H), 1.46 (s, 9H), 1.36 (qd, J=4.3, 12.6 Hz, 2H).

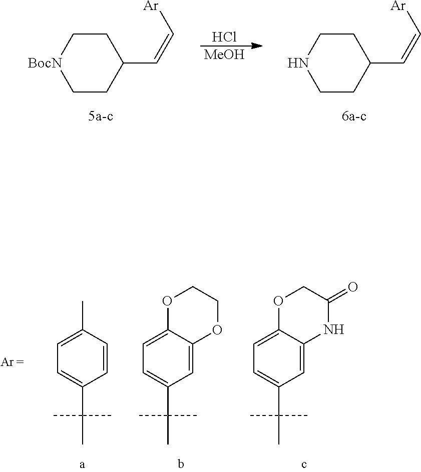

(Z)-4-(4-methylstyryl)piperidine*hydrogen chloride (6a): Title compound was synthesized from piperidine 5a using General Procedure 2. No purification or isolation attempted. Crude compounds used directly in next step.

(Z)-4-(2-(2,3-dihydrobenzo[b][1,4]dioxin-6-yl)vinyl)piperidine*hydrogen chloride (6b): Title compound was synthesized from piperidine 5b using General Procedure 2. No purification or isolation attempted. Crude compounds used directly in next step.

(Z)-6-(2-(piperidin-4-yl)vinyl)-2H-benzo[b][1,4]oxazin-3(4H)-one*hydrogen chloride (6c): Title compound was synthesized from piperidine 5c using General Procedure 2. No purification or isolation attempted. Crude compounds used directly in next step.

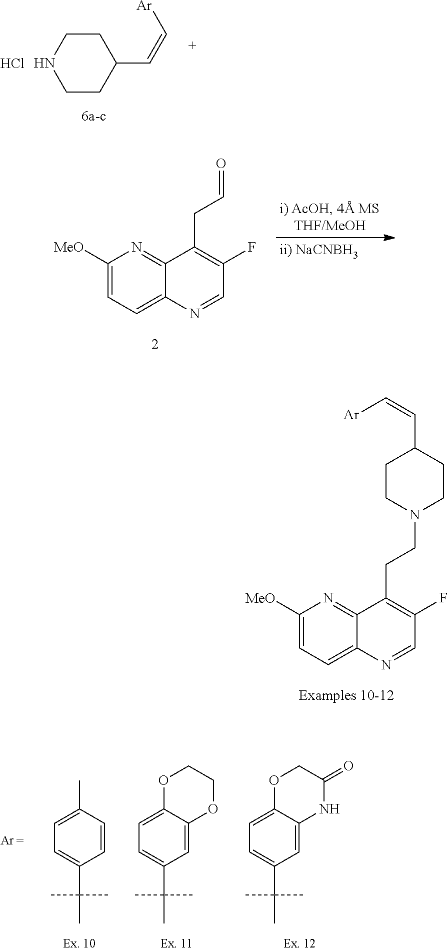

Example 10. (Z)-7-fluoro-2-methoxy-8-(2-(4-(4-methylstyryl)piperidin-1-yl)ethyl)-1,5-naphthyridine: Title compound was synthesized from (Z)-olefin 6a and aldehyde 2 using General Procedure 3. Purification by flash chromatography (compound eluted in 10% ethyl acetate in hexanes) and concentration in vacuo afforded the title compound as a yellow oil (91 mg, 0.224 mmol, 62% yield).

¹HNMR (DMSO-d₆, 400 MHz) δ: 8.77 (s, 1H), 8.27 (d, J=9.0 Hz, 1H), 7.23 (d, J=9.1 Hz, 1H), 7.16 (d, J=8.5 Hz, 2H), 7.13 (d, J=8.6 Hz, 2H), 6.30 (d, J=11.6 Hz, 1H), 5.42 (dd, J=10.2, 11.2 Hz, 1H), 4.03 (s, 3H), 3.31-3.28 (m, 3H) 2.98-2.95 (m, 2H), 2.68-2.63 (m, 2H), 2.28 (s, 3H), 2.07-2.02 (m, 2H), 1.62-1.59 (m, 2H), 1.40-1.30 (m, 2H). ¹³CNMR (DMSO-d₆, 100 MHz) δ: 161.98, 156.80 (d, J=254.3 Hz), 140.90 (d, J=7.0 Hz), 140.39, 138.08 (d, J=1.4 Hz), 137.85 (d, J=27.8 Hz), 136.59, 135.89, 134.19, 128.91, 128.17, 127.54, 115.27, 56.87, 53.55, 52.41, 34.65, 31.96, 20.68, 20.60. HRMS (ESI) m/z calc'd for C₂₅H₂₉FN₃O [M+H]⁺: 406.22892; found: 406.22882. UPLC: rt: 3.92 min, purity: 97.7%.
Example 11. (Z)-8-(2-(4-(2-(2,3-dihydrobenzo[b][1,4]dioxin-6-yl)vinyl)piperidin-1-yl)ethyl)-7-fluoro-2-methoxy-1,5-naphthyridine: Title compound was synthesized from (Z)-olefin 6b and aldehyde 2 using General Procedure 3. Purification by flash chromatography (compound eluted in 10% ethyl acetate in hexanes) and concentration in vacuo afforded the title compound as a clear oil (197 mg, 0.438 mmol, 58% yield).

¹HNMR (DMSO-d₆, 400 MHz) δ: 8.77 (s, 1H), 8.28 (d, J=9.0 Hz, 1H), 7.23 (d, J=9.0 Hz, 1H), 6.82 (dd, J=2.3, 6.6 Hz, 1H), 6.74-6.71 (m, 3H), 6.21 (d, J=11.7 Hz, 1H), 5.35 (dd, J=10.2, 11.2 Hz, 1H), 4.23 (s, 4H), 4.04 (s, 3H), 3.35-3.28 (obscured by water, m, 2H), 3.02-2.94 (m, 2H), 2.72-2.64 (m, 2H), 2.12-2.01 (m, 2H), 1.65-1.57 (m, 2H), 1.40-1.27 (m, 2H). ¹³CNMR (DMSO-d₆, 100 MHz) δ: 162.00, 156.81 (d, J=254.2 Hz), 143.02, 142.32, 140.90 (d, J=7.0 Hz), 140.41, 138.09 (d, J=2.0 Hz), 137.87 (d, J=27.8 Hz), 135.87, 130.34, 127.09 121.49, 116.90, 116.66, 115.25 (d, J=12.8 Hz), 64.04 (d, J=5.3 Hz), 56.82, 53.59, 52.38, 34.58, 31.89, 20.55. HRMS (ESI) m/z calc'd for C₂₆H₂₉FN₃O₃ [M+H]⁺: 450.21875; found: 450.21750. UPLC: rt: 3.70 min, purity: 90.6%.
Example 12. (Z)-4-benzyl-6-(2-(1-(2-(3-fluoro-6-methoxy-1,5-naphthyridin-4-yl)ethyl)piperidin-4-yl)vinyl)-2H-benzo[b][1,4]oxazin-3(4H)-one: Title compound was synthesized from (Z)-olefin 6c and aldehyde 2 using General Procedure 3. Purification by flash chromatography (compound eluted in 50% ethyl acetate in hexanes) and concentration in vacuo afforded the title compound as a yellow solid (20 mg, 0.043 mmol, 38% yield).

¹HNMR (CDCl₃, 400 MHz, contaminated w/ ethyl acetate) δ: 8.61 (s, 1H), 8.52 (s, 1H), 8.17 (d, J=9.0 Hz, 1H), 7.07 (d, J=9.0 Hz, 1H), 6.94 (d, J=8.3 Hz, 1H), 6.87 (dd, J=1.6, 8.3 Hz, 1H), 6.69 (d, J=1.6 Hz, 1H), 6.27 (d, J=11.6 Hz, 1H), 5.48 (dd, J=10.1, 11.4 Hz, 1H), 4.62 (s, 2H), 4.07 (s, 3H), 3.43-3.37 (m, 2H), 3.10-3.04 (m, 2H), 2.75-2.71 (m, 2H), 2.58-2.47 (m, 1H), 2.17-2.11 (m, 2H), 1.73-1.69 (m, 2H), 1.59-1.49 (m, 2H). ¹³CNMR (CDCl₃, 100 MHz) δ: 165.83, 162.54, 157.43 (d, J=255.2 Hz), 142.49, 141.80 (d, J=6.8 Hz), 140.29, 138.66 (d, J=2.4 Hz), 138.04 (d, J=28.0 Hz), 137.67, 132.81, 130.75 (d, J=13.1 Hz), 126.83, 125.99, 124.56, 116.75, 115.98, 115.34 (d, J=2.6 Hz), 67.48, 57.67, 53.94, 53.15, 35.21, 32.58, 21.10. HRMS (ESI) m/z calc'd for C₂₆H₂₆FN₄O₃ [M+H]⁺: 463.21400; found: 463.21304. UPLC: rt: 3.38 min, purity: 97.7%.

The Sharpless asymmetric dihydroxylation of (Z)-alkenes typically proceeds with poor levels of enantioselectivity (Wang, L. and Sharpless, K. B. J. Am. Chem. Soc. 1992, 114. 7568-7570). The absolute stereochemistry of Examples 13-15 and 16-18 was not determined.

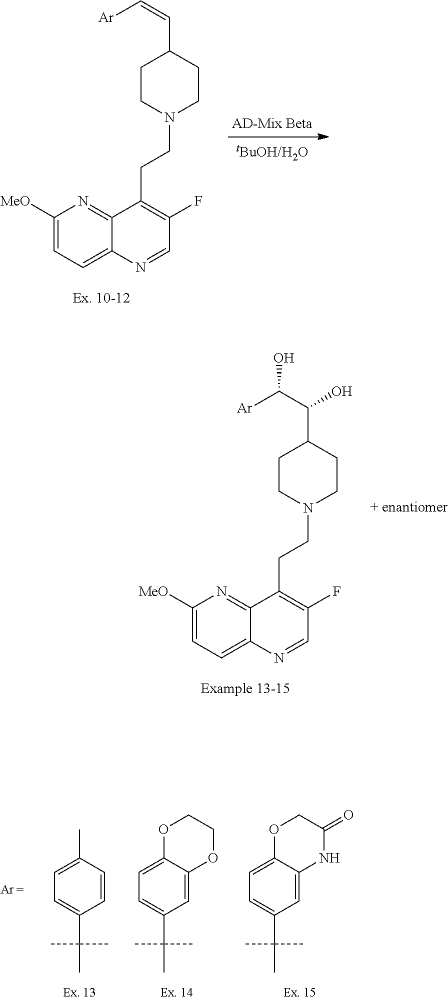

Example 13. 1-(1-(2-(3-fluoro-6-methoxy-1,5-naphthyridin-4-yl)ethyl)piperidin-4-yl)-2-(p-tolyl)ethane-1,2-diol: Title compound was synthesized from Example 10 using General Procedure 9 with AD-mix β. Purification by flash chromatography (compound eluted in 100% ethyl acetate) and concentration in vacuo afforded the title compound (14 mg, 0.033 mmol, 87% yield).

¹HNMR (DMSO-d₆, 400 MHz) δ: 8.76 (s, 1H), 8.27 (d, J=9.0 Hz, 1H), 7.25-7.19 (m, 3H), 7.07 (d, J=7.9 Hz, 2H), 5.03 (d, J=4.9 Hz, 1H), 4.31 (dd, J=6.7, 5.1 Hz, 1H), 4.15 (d, J=6.1 Hz, 1H), 4.04 (s, 3H), 3.31-3.25 (m, 3H, partially obscured by water), 3.00 (br t, J=11.6 Hz, 2H), 2.67-2.58 (m, 2H), 2.27 (s, 3H), 1.98-1.86 (m, 2H), 1.69-1.61 (m, 1H), 1.55-1.45 (m, 1H), 1.44-1.37 (m, 2H), 1.37-1.25 (m, 1H). ¹³CNMR (DMSO-d&, 100 MHz) δ: 161.95, 156.83 (d, J=254.0 Hz), 141.29, 140.93 (d, J=7.0 Hz), 140.40, 138.08 (d, J=1.4 Hz), 137.86 (d, J=27.8), 135.37, 130.26, 130.14 127.95, 127.36, 115.27, 77.14, 73.29, 56.95 53.54, 53.45, 53.27, 37.27, 29.11, 25.38, 20.69. HRMS (ESI) m/z calc'd for C₂₅H₃₁FN₃O₃ [M+H]⁺: 440.2344; found: 440.23337. UPLC: rt: 3.259 min, purity: 93.4%. Enantiomeric excess (ee) undetermined.
Example 14. 1-(2,3-dihydrobenzo[b][1,4]dioxin-6-yl)-2-(1-(2-(3-fluoro-6-methoxy-1,5-naphthyridin-4-yl)ethyl)piperidin-4-yl)ethane-1,2-diol: Title compound was synthesized from Example 11 using General Procedure 9 with AD-mix β. Purification by flash chromatography (compound eluted in 2% methanol in DCM) and concentration in vacuo afforded the title compound as a white solid (8 mg, 0.016 mmol, 11% yield).

¹HNMR (DMSO-d₆, 400 MHz; contaminated w/ DMSO) δ: 8.76 (s, 1H), 8.27 (d, J=9.0 Hz, 1H), 7.23 (d, J=9.0 Hz, 1H), 6.82 (d, J=1.8 Hz, 1H), 6.77 (dd, J=1.8, 8.3 Hz, 1H), 6.73 (d, J=8.2 Hz, 1H), 5.00 (d, J=4.9 Hz, 1H). 4.24-4.20 (m, 1H), 4.21 (s, 4H), 4.15 (d, J=6.1 Hz, 1H), 4.04 (s, 3H), 3.31-3.27 (m, 2H, obscured by water), 3.26-3.21 (m, 1H), 3.05-2.96 (m, 2H), 2.65-2.61 (m, 2H), 1.98-1.87 (m, 2H), 1.66-1.60 (m, 1H), 1.54-1.45 (m, 1H), 1.43-1.39 (m, 2H), 1.37-1.23 (m, 1H). ¹³CNMR (DMSO-d₆, 100 MHz, contaminated w/ DMSO) δ: 161.96, 156.84 (d, J=254.0 Hz), 142.50, 142.02, 140.94 (d, J=7.1 Hz), 140.41, 138.08 (d, J=1.9 Hz), 137.86 (d, J=27.9 Hz), 137.48, 130.21 (d, J=13.0 Hz), 120.26, 116.08, 115.81, 115.27 (d, J=2.4 Hz), 77.11, 72.94, 64.00, 56.95, 53.55, 53.46, 53.27, 37.27, 29.09, 25.36, 20.72. HRMS (ESI) m/z calc'd for C₂₆H₃₁FN₃O₅ [M+H]⁺: 484.22342; found: 484.22342. UPLC: rt: 3.035 min, purity: 99%. Enantiomeric excess (ee) undetermined.
Example 15. 6-(-2-(1-(2-(3-fluoro-6-methoxy-1,5-naphthyridin-4-yl)ethyl)piperidin-4-yl)-1,2-dihydroxyethyl)-2H-benzo[b][1,4]oxazin-3(4H)-one: Title compound was synthesized from Example 12 using General Procedure 9 with AD-mix β. Purification by flash chromatography (compound eluted in 1% methanol in DCM) and concentration in vacuo afforded the title compound as a white solid (20 mg, 0.040 mmol, 25% yield).

¹HNMR (DMSO-d₆, 400 MHz) δ: 10.64 (s, 1H), 8.77 (s, 1H), 8.27 (d, J=9.0 Hz, 1H), 7.23 (d, J=9.0 Hz, 1H), 6.92 (d, J=1.6 Hz, 1H), 6.87 (dd, J=1.7, 8.3 Hz, 1H), 6.83 (d, J=8.2 Hz, 1H), 5.13 (d, J=4.4 Hz, 1H), 4.51 (s, 2H), 4.23 (dd, J=4.4, 7.1 Hz, 2H), 4.04 (s, 3H), 3.34-3.28 (m, 2H, obscured by water), 3.26-3.20 (m, 1H), 3.11-2.99 (m, 2H), 2.74-2.60 (m, 2H), 2.10-1.89 (m, 2H), 1.69-1.62 (m, 1H), 1.62-1.52 (m, 1H), 1.51-1.29 (m, 3H). ¹³CNMR (DMSO-d₆, 100 MHz) δ: 164.97, 161.98, 156.81 (d, J=254.2 Hz), 141.97, 140.89 (d, J=7.1 Hz), 140.40, 138.88, 138.08 (d, J=1.2 Hz), 137.86 (d, J=27.8 Hz), 126.37, 122.07, 115.28 (d, J=1.8 Hz), 114.98, 77.01, 72.94, 66.77, 56.75, 53.56, 53.34, 53.17, 37.09, 28.93, 24.99, 20.55. HRMS (ESI) m/z calc'd for C₂₆H₃₀FN₄O₅ [M+H]⁺: 497.21947; found: 497.21863. UPLC: rt: 2.867 min, purity: 97.8%. Enantiomeric excess (ee) undetermined.

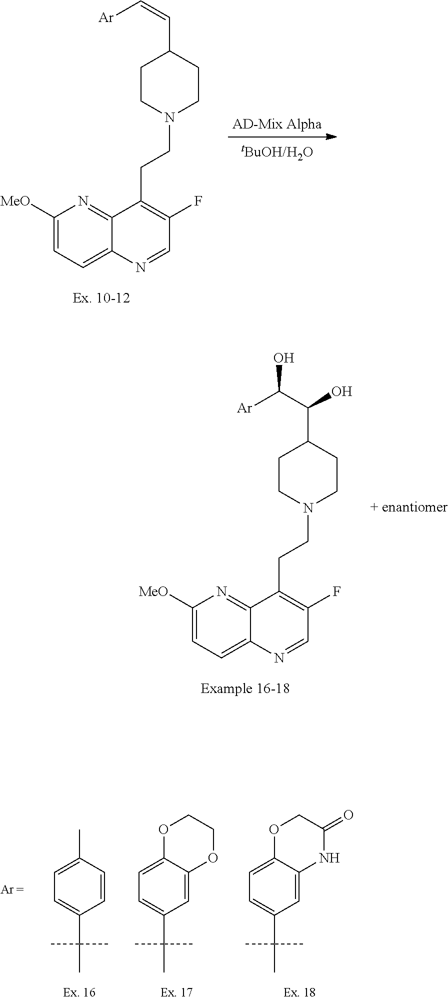

Example 16. 1-(1-(2-(3-fluoro-6-methoxy-1,5-naphthyridin-4-yl)ethyl)piperidin-4-yl)-2-(p-tolyl)ethane-1,2-diol: Title compound was synthesized from Example 10 using General Procedure 9 with AD-mix α. Purification by flash chromatography (compound eluted in 1% methanol in DCM) and concentration in vacuo afforded the title compound white solid (14 mg, 0.032 mmol, 16% yield).

¹HNMR (DMSO-d₆, 400 MHz, contaminated w/ DCM) δ: 8.76 (s, 1H), 8.27 (d, J=9.0 Hz, 1H), 7.23 (d, J=9.0 Hz, 1H), 7.21 (d, J=8.0 Hz, 2H), 7.07 (d, J=7.8 Hz, 2H), 5.04 (d, J=4.8 Hz, 1H), 4.31 (dd, J=5.0, 6.9 Hz, 1H), 4.15 (d, J=6.1 Hz, 1H), 4.04 (s, 3H), 3.32-3.27 (m, 3H, partially obscured by water), 3.05-2.95 (m, 2H), 2.65-2.59 (m, 2H), 2.27 (s, 3H), 1.99-1.86 (m, 2H), 1.68-1.61 (m, 1H), 1.56-1.45 (m, 1H), 1.44-1.37 (m, 2H), 1.37-1.25 (m, 2H). ¹³CNMR (DMSO-d₆, 100 MHz) δ: 161.95, 156.82 (d, J=254.2 Hz), 141.29, 140.93 (d, J=7.1 Hz), 140.40, 138.08 (d, J=1.4 Hz), 137.86 (d, J=27.8), 135.37, 130.25, 130.12, 127.95, 127.36, 115.27, 77.14, 73.29, 56.95 53.54, 53.45, 53.27, 37.27, 29.11, 25.38, 20.69. HRMS (ESI) m/z calc'd for C₂₅H₃₁FN₃O₃ [M+H]⁺: 440.2344; found: 440.23389. UPLC: rt: 3.188 min, purity: 95.9%. Enantiomeric excess (ee) undetermined.
Example 17. 1-(2,3-dihydrobenzo[b][1,4]dioxin-6-yl)-2-(1-(2-(3-fluoro-6-methoxy-1,5-naphthyridin-4-yl)ethyl)piperidin-4-yl)ethane-1,2-diol: Title compound was synthesized from Example 11 using General Procedure 9 with AD-mix α. Purification by flash chromatography (compound eluted in 2% methanol in DCM) and concentration in vacuo afforded the title compound as a white solid (7 mg, 0.015 mmol, 11% yield).

¹HNMR (DMSO-d₆, 400 MHz; contaminated w/ DCM) δ: 8.76 (s, 1H), 8.27 (d, J=9.0 Hz, 1H), 7.23 (d, J=9.0 Hz, 1H), 6.82 (d, J=1.8 Hz, 1H), 6.77 (dd, J=1.8, 8.3 Hz, 1H), 6.73 (d, J=8.2 Hz, 1H), 5.00 (d, J=4.9 Hz, 1H). 4.24-4.20 (m, 1H), 4.20 (s, 4H), 4.15 (d, J=6.1 Hz, 1H), 4.04 (s, 3H), 3.31-3.27 (m, 2H, obscured by water), 3.26-3.21 (m, 1H), 3.05-2.96 (m, 2H), 2.65-2.61 (m, 2H), 1.98-1.87 (m, 2H), 1.66-1.60 (m, 1H), 1.54-1.45 (m, 1H), 1.43-1.39 (m, 2H), 1.36-1.23 (m, 1H). ¹³CNMR (DMSO-d₆, 100 MHz, contaminated w/ DMSO) δ: 161.96, 156.84 (d, J=254.0 Hz), 142.50, 142.02, 140.94 (d, J=7.1 Hz), 140.41, 138.08 (d, J=1.9 Hz), 137.86 (d, J=27.9 Hz), 137.48, 130.21 (d, J=13.0 Hz), 120.26, 116.08, 115.81, 115.27 (d, J=2.4 Hz), 77.11, 72.94, 64.00, 56.95, 53.55, 53.46, 53.27, 37.27, 29.09, 25.36, 20.72. HRMS (ESI) m/z calc'd for C₂₆H₃₁FN₃O₅ [M+H]⁺: 484.22342; found: 484.22305. UPLC: rt: 3.037 min, purity: 96.6%. Enantiomeric excess (ee) undetermined.
Example 18. 6-((1R,2S)-2-(1-(2-(3-fluoro-6-methoxy-1,5-naphthyridin-4-yl)ethyl)piperidin-4-yl)-1,2-dihydroxyethyl)-2H-benzo[b][1,4]oxazin-3(4H)-one: Title compound was synthesized from Example 12 using General Procedure 9 with AD-mix β. Purification by flash chromatography (compound eluted in 1% methanol in DCM) and concentration in vacuo afforded the title compound as a white solid (20 mg, 0.040 mmol, 25% yield).

¹HNMR (DMSO-d₆, 400 MHz) δ: 10.64 (s, 1H), 8.77 (s, 1H), 8.27 (d, J=9.0 Hz, 1H), 7.23 (d, J=9.0 Hz, 1H), 6.92 (d, J=1.6 Hz, 1H), 6.87 (dd, J=1.7, 8.3 Hz, 1H), 6.83 (d, J=8.2 Hz, 1H), 5.13 (d, J=4.4 Hz, 1H), 4.51 (s, 2H), 4.23 (dd, J=4.4, 7.1 Hz, 2H), 4.04 (s, 3H), 3.34-3.28 (m, 2H, obscured by water), 3.26-3.20 (m, 1H), 3.08-2.96 (m, 2H), 2.68-2.60 (m, 2H), 2.10-1.89 (m, 2H), 1.69-1.62 (m, 1H), 1.62-1.52 (m, 1H), 1.51-1.28 (m, 3H). ¹³CNMR (DMSO-d₆, 100 MHz) δ: 164.97, 161.98, 156.81 (d, J=254.2 Hz), 141.97, 140.89 (d, J=7.1 Hz), 140.40, 138.88, 138.08 (d, J=1.2 Hz), 137.86 (d, J=27.8 Hz), 126.37, 122.07, 115.28 (d, J=1.8 Hz), 114.98, 77.01, 72.94, 66.77, 56.75, 53.56, 53.34, 53.17, 37.09. 28.93, 24.99, 20.55. HRMS (ESI) m/z calc'd for C₂₆H₃₀FN₄O₅ [M+H]⁺: 497.21947; found: 497.21942. UPLC: rt: 2.866 min, purity: 94.3%. Enantiomeric excess (ee) undetermined.

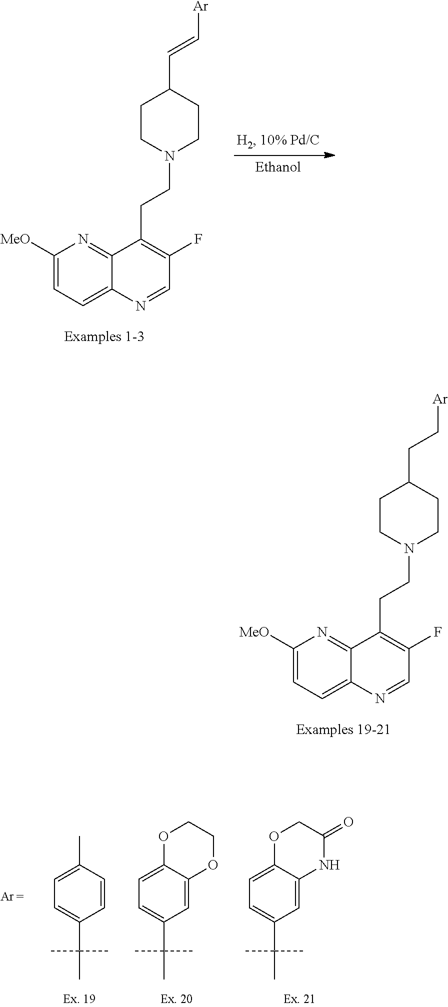

Example 19. 7-fluoro-2-methoxy-8-(2-(4-(4-methylphenethyl)piperidin-1-yl)ethyl)-1,5-naphthyridine: Title compound was synthesized from Example 1 using General Procedure 8. Purification by flash chromatography (compound eluted in 20% ethyl acetate in hexanes) and concentration in vacuo afforded the title compound (4 mg, 0.010 mmol, 16% yield).

¹HNMR (CDCl₃, 400 MHz) δ: 8.61 (s, 1H), 8.17 (d, J=9.0 Hz, 1H), 7.12-7.03 (m, 5H), 4.08 (s, 3H), 3.45-3.36 (m, 2H), 3.12-3.04 (m, 2H), 2.76-2.67 (m, 2H), 2.64-2.55 (m, 2H), 2.32 (s, 3H), 2.16-2.03 (m, 2H), 1.83-1.72 (m, 2H), 1.61-1.51 (m, 2H), 1.40-1.27 (m, 3H). ¹³CNMR (CDCl₃, 100 MHz) δ: 162.53, 157.42 (d, J=255.3 Hz), 141.82 (d, J=7.0 Hz), 140.28, 139.84, 138.65 (d, J=2.1 Hz), 138.07 (d, J=28.1 Hz), 135.21, 130.85 (d, J=12.9 Hz), 129.14, 126.34, 115.31 (d, J=2.8 Hz), 57.67, 53.97, 53.84, 38.70, 35.45, 32.78, 32.58, 32.42, 21.13, 21.10 (d, J=1.0 Hz). HRMS (ESI) m/z calc'd for C₂₅H₃FN₃O [M+H]⁺: 408.24457; found: 408.24403. UPLC: rt: 3.97 min, purity: 96.9%.
Example 20. 8-(2-(4-(2-(2,3-dihydrobenzo[b][1,4]dioxin-6-yl)ethyl)piperidin-1-yl)ethyl)-7-fluoro-2-methoxy-1,5-naphthyridine: Title compound was synthesized from Example 2 using General Procedure 8. Purification by flash chromatography (compound eluted in 15% ethyl acetate in hexanes) and concentration in vacuo afforded the title compound (15 mg, 0.033 mmol, 17% yield).

¹HNMR (DMSO-d₆, 400 MHz, contaminated w/ DCM) δ: 8.76 (s, 1H), 8.27 (d, J=9.0 Hz, 1H), 7.23 (d, J=9.0 Hz, 1H), 6.71 (d, J=8.1 Hz), 6.64 (d, J=1.8 Hz, 1H), 6.60 (dd, J=1.9, 8.2 Hz, 1H), 4.18 (s, 4H), 3.31-3.27 (m, 2H), 2.97-2.94 (m, 2H), 2.68-2.60 (m, 2H), 2.46-2.42 (m, 2H), 2.02-1.92 (m, 2H), 1.68-1.61 (m, 2H), 1.44-1.37 (m, 2H), 1.22-1.03 (m, 3H). ¹³CNMR (DMSO-d₆, 100 MHz) δ: 161.97, 156.80 (d, J=254.2 Hz), 143.02, 141.25, 140.89 (d, J=6.8 Hz), 140.39, 138.07 (d, J=1.6 Hz), 137.85 (d, J=27.8 Hz), 135.34, 120.80, 116.65, 116.52, 115.27, 63.95 (d, J=11.1 Hz), 56.83, 53.52, 53.02, 37.98, 34.65, 31.90, 31.50, 20.62. HRMS (ESI) m/z calc'd for C₂H₃₁FN₃O₃ [M+H]⁺: 452.2344; found: 452.23306. UPLC: rt: 3.694 min, purity: 98.0%.
Example 21. 6-(2-(1-(2-(3-fluoro-6-methoxy-1,5-naphthyridin-4-yl)ethyl)piperidin-4-yl)ethyl)-2H-benzo[b][1,4]oxazin-3(4H)-one: Title compound was synthesized from Example 3 using General Procedure 8. Purification by flash chromatography (compound eluted in 100% DCM) and concentration in vacuo afforded the title compound off-white solid (3 mg, 0.006 mmol, 6% yield).

¹HNMR (CDCl₃, 400 MHz) δ: 8.61 (s, 1H), 8.17 (d, J=9.0 Hz, 1H), 7.99 (s, 1H), 7.07 (d, J=9.0 Hz, 1H), 6.89 (d, J=8.2 Hz, 1H), 6.79 (dd, J=1.9, 8.2 Hz, 1H), 6.60 (d, J=1.9 Hz, 1H), 4.59 (s, 2H), 4.08 (s, 3H), 3.43-3.38 (m, 2H), 3.11-3.05 (m, 2H), 2.76-2.70 (m, 2H), 2.59-2.53 (m, 2H), 2.13-2.05 (m, 2H), 1.78-1.71 (m, 2H), 1.57-1.50 (m, 2H), 1.37-1.25 (m, 3H). ¹³CNMR (CDCl₃, 100 MHz) δ: 165.81, 162.54, 157.43 (d, J=255.3 Hz), 141.86, 141.79, 140.30, 138.67 (d, J=2.1 Hz), 138.07 (d, J=28.1 Hz), 137.76, 130.82 (d, J=12.9 Hz), 126.02, 124.09, 116.82, 115.66, 115.32 (d, J=2.8 Hz), 67.57, 57.64, 53.95, 53.81, 38.62, 35.48, 32.55, 32.52, 21.12. HRMS (ESI) m/z calc'd for C₆H₃₀FN₄O₃ [M+H]⁺: 465.22965; found: 465.22867. UPLC: rt: 3.369 min, purity: 96.8%.

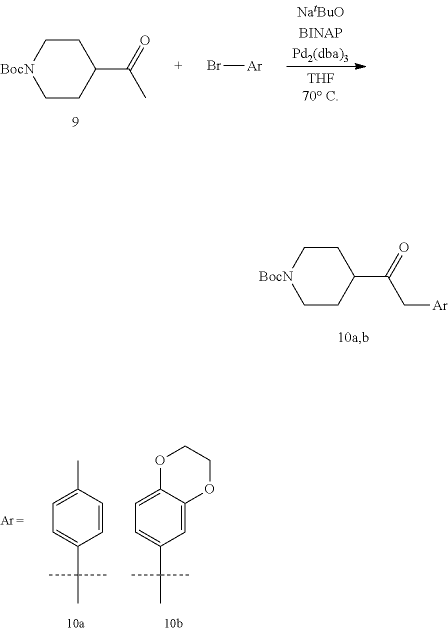

tert-butyl 4-(2-(p-tolyl)acetyl)piperidine-1-carboxylate (10a): Title compound was synthesized from methyl ketone 9 and 1-bromo-4-methylbenzene using General Procedure 1. Purification by flash chromatography (compound eluted in 10% ethyl acetate in hexanes) and concentration in vacuo afforded the title compound (260 mg, 0.819 mmol, 82% yield).

¹HNMR (CDCl₃, 400 MHz) δ: 7.13 (d, J=8.0 Hz, 2H), 7.07 (d, J=8.0 Hz, 2H), 4.12-4.00 (m, 2H), 3.70 (s, 2H), 2.73 (t, J=12.3 Hz, 2H), 2.58 (tt, J=3.8, 11.3 Hz, 1H), 2.33 (s, 3H), 1.80-1.71 (m, 2H), 1.60-1.49 (m, 2H, obscured by water), 1.44 (s, 9H).

tert-butyl 4-(2-(2,3-dihydrobenzo[b][1,4]dioxin-6-yl)acetyl)piperidine-1-carboxylate (10b): Title compound was synthesized from methyl ketone 9 and 6-bromo-2,3-dihydrobenzo[b][1,4]dioxine using General Procedure 1. Purification by flash chromatography (compound eluted in 15% ethyl acetate in hexanes) and concentration in vacuo afforded the title compound as a yellow oil (180 mg, 0.498 mmol, 50% yield).

¹HNMR (CDCl₃, 400 MHz, contaminated w/ ethyl acetate and acetone) δ: 6.81 (d, J=8.2 Hz, 1H), 6.69 (d, J=2.0 Hz, 1H), 6.64 (dd, J=2.1, 8.2 Hz, 1H), 4.24 (s, 4H), 4.13-4.02 (m, 2H), 3.62 (s, 2H), 2.77-2.70 (m, 2H), 2.57 (tt, J=3.7, 11.2 Hz, 1H), 1.60-1.48 (m, 4H, obscured by water), 1.45 (s, 9H).

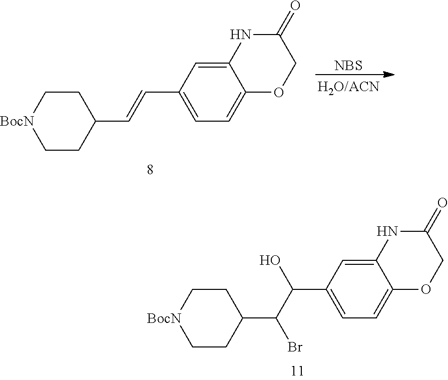

tert-butyl 4-(1-bromo-2-hydroxy-2-(3-oxo-3,4-dihydro-2H-benzo[b][1,4]oxazin-6-yl)ethyl)piperidine-1-carboxylate (11): Title compound was synthesized from (E)-olefin 8 using General Procedure 12. Purification by flash chromatography (compound eluted in 40% ethyl acetate in hexanes) and concentration in vacuo afforded the title compound as a white solid (180 mg, 0.395 mmol, 71% yield).

¹HNMR (CDCl₃, 400 MHz, contaminated w/ ethyl acetate and DCM) δ: 7.67 (s, 1H), 7.04-6.89 (m, 2H), 6.87 (d, J=1.4 Hz, 1H), 4.89 (dd, J=3.3, 7.5 Hz, 1H), 4.66 (s, 2H), 4.18-4.14 (m, 2H, obscured by ethyl acetate), 2.78-2.66 (m, 2H), 2.25 (d, J=3.3 Hz, 1H), 2.05-1.96 (m, 1H), 1.92-1.90 (m, 1H), 1.70-1.56 (m, 2H, obscured by water), 1.56-1.48 (m, 2H, obscured by water and next peak), 1.48 (s, 9H).

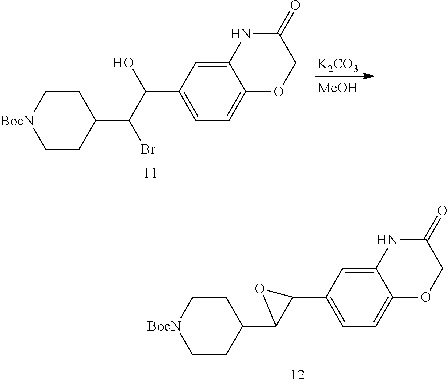

tert-butyl 4-(3-(3-oxo-3,4-dihydro-2H-benzo[b][1,4]oxazin-6-yl)oxiran-2-yl)piperidine-1-carboxylate (12): Title compound was synthesized from bromohydrin 10 using General Procedure 13. No purification performed. Title compound was afforded as a yellow solid (200 mg, 0.385 mmol, 100% yield).

¹HNMR (CDCl₃, 400 MHz, contaminated w/ ethyl acetate) δ: 6.96-6.85 (m, 2H), 6.62 (s, 1H), 4.59 (s, 2H), 4.21-4.07 (m, 2H, obscured by ethyl acetate), 3.63 (s, 1H), 2.78-2.65 (m, 3H), 1.89-1.80 (m, 1H), 1.73-1.65 (m, 1H), 1.59-1.50 (m, 1H), 1.46 (s, 9H), 1.42-1.28 (m, 4H, obscured by ethyl acetate).

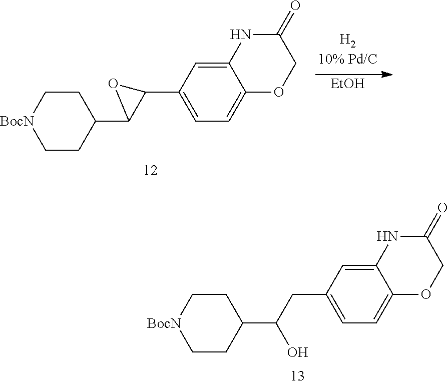 [SMILES: "EtOH" ; CC(C)(C)OC(=O)N1CCC(C2OC2c2ccc3c(c2)NC(=O)CO3)CC1 ; CC(C)(C)OC(=O)N1CCC(C(O)Cc2ccc3c(c2)NC(=O)CO3)CC1]

tert-butyl 4-(1-hydroxy-2-(3-oxo-3,4-dihydro-2H-benzo[b][1,4]oxazin-6-yl)ethyl)piperidine-1-carboxylate (13): Title compound was synthesized from epoxide 12 using General Procedure 14. No purification performed. Title compound afforded as a white solid (140 mg, 0.372 mmol, 97% yield).

¹HNMR (CDCl₃, 400 MHz, contaminated w/ DCM) δ: 6.93 (d, J=8.2 Hz, 1H), 6.84-6.80 (m, 1H), 6.67-6.64 (m, 1H), 4.61 (s, 2H), 4.23-4.12 (m, 2H), 3.59-3.51 (m, 1H), 2.85-2.77 (m, 1H), 2.73-2.61 (m, 2H), 2.57-2.48 (m, 1H), 1.89-1.81 (m, 1H), 1.71-1.64 (m, 1H), 1.60-1.51 (m, 1H), 1.46 (s, 9H), 1.43-1.27 (m, 4H).

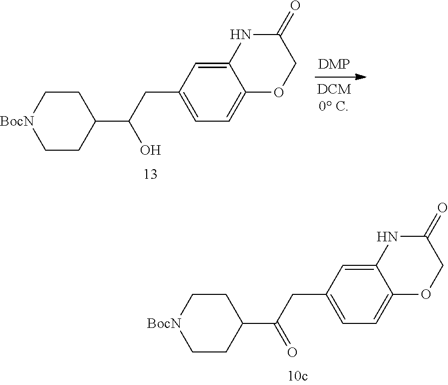 [SMILES: CC(C)(C)OC(=O)N1CCC(C(O)Cc2ccc3c(c2)NC(=O)CO3)CC1 ; CC(C)(C)OC(=O)N1CCC(C(=O)Cc2ccc3c(c2)NC(=O)CO3)CC1]

tert-butyl 4-(2-(3-oxo-3,4-dihydro-2H-benzo[b][1,4]oxazin-6-yl)acetyl)piperidine-1-carboxylate (10c): Title compound was synthesized from hydroxyl 13 using General Procedure 15. No purification performed. Title compound afforded as a clear oil (110 mg, 0.278 mmol, 100% yield).

¹HNMR (CDCl₃, 400 MHz, contaminated w/ ethyl acetate) δ: 7.77 (s, 1H), 6.93 (d, J=8.2 Hz, 1H), 6.77 (dd, J=2.0, 8.2 Hz, 1H), 6.62 (d, J=2.0 Hz, 111), 4.61 (s, 2H), 4.14-4.04 (m, 2H, obscured by ethyl acetate), 3.68 (s, 2H), 2.81-2.72 (m, 2H), 2.57 (tt, J=3.8, 11.4 Hz, 1H), 1.84-1.75 (m, 2H), 1.61-1.50 (m, 2H, obscured by water), 1.46-1.43 (m, 9H).

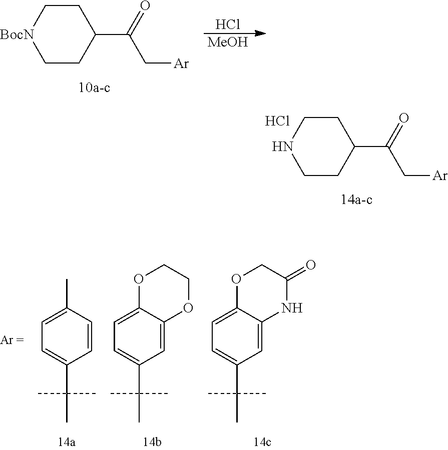

1-(piperidin-4-yl)-2-(p-tolyl)ethan-1-one*hydchloride (14a): Title compound was synthesized from piperidine 10a using General Procedure 2. No purification or isolation attempted. Crude compounds used directly in next step.

2-(2,3-dihydrobenzo[b][1,4]dioxin-6-yl)-1-(piperidin-4-yl)ethan-1-one*hydrochloride (14b): Title compound was synthesized from piperidine 10b using General Procedure 2. No purification or isolation attempted. Crude compounds used directly in next step.

6-(2-oxo-2-(piperidin-4-yl)ethyl)-2H-benzo[b][1,4]oxazin-3(4H)-one*hydrochloride (14c): Title compound was synthesized from piperidine 10c using General Procedure 2. No purification or isolation attempted. Crude compounds used directly in next step.

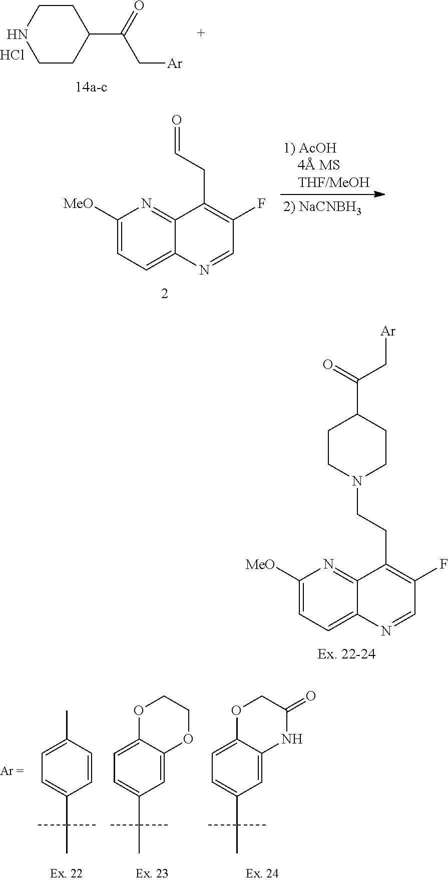

Example 22. 1-(1-(2-(3-fluoro-6-methoxy-1,5-naphthyridin-4-yl)ethyl)piperidin-4-yl)-2-(p-tolyl)ethan-1-one: Title compound was synthesized from piperidine 14a and aldehyde 2 using General Procedure 3. Purification by flash chromatography (compound eluted in 20% ethyl acetate in hexanes) and concentration in vacuo afforded the title compound as a yellow solid (175 mg, 0.415 mmol, 65% yield).

¹HNMR (DMSO-d₆, 400 MHz) δ: 8.77 (s, 1H), 8.27 (d, J=9.0 Hz, 1H), 7.23 (d, J=9.0 Hz, 1H), 7.10 (d, J=7.8 Hz, 2H), 7.04 (d, J=8.0 Hz, 2H), 4.04 (s, 3H), 3.75 (s, 2H), 3.31-3.27 (m, 3H, obscured by water), 3.04-2.94 (m, 2H), 2.73-2.63 (m, 2H), 2.26 (s, 3H), 2.14-2.00 (m, 2H), 1.81-1.73 (m, 2H), 1.48-1.35 (m, 2H). ¹³CNMR (DMSO-d₆, 100 MHz) δ: 210.06, 162.00. 156.82 (d, J=254.2 Hz), 140.88 (d, J=7.0 Hz), 140.42, 138.08 (d, J=2.0 Hz), 137.88 (d, J=27.8 Hz), 135.40, 131.73, 129.63, 129.44, 128.80, 128.18, 115.31 (d, J=2.5 Hz), 56.52, 53.58, 52.21, 47.24, 46.34, 27.38, 20.62, 20.49. HRMS (ESI) m/z calc'd for C₂₅H₂₉FN₃O₂ [M+H]⁺: 422.22383; found: 422.22302. UPLC: rt: 3.612 min, purity: 91.9%.
Example 23. 2-(2,3-dihydrobenzo[b][1,4]dioxin-6-yl)-1-(1-(2-(3-fluoro-6-methoxy-1,5-naphthyridin-4-yl)ethyl)piperidin-4-yl)ethan-1-one: Title compound was synthesized from piperidine 14b and aldehyde 2 using General Procedure 3. Purification by flash chromatography (compound eluted in 15% ethyl acetate in hexanes) and concentration in vacuo afforded the title compound as a yellow solid (55 mg, 0.118 mmol, 22% yield).

¹HNMR (DMSO-d₆, 400 MHz) δ: 8.77 (s, 1H), 8.28 (d, J=9.0 Hz, 1H), 7.24 (d, J=9.0 Hz, 1H), 6.75 (d, J=8.2 Hz, 1H), 6.65 (d, J=2.0 Hz, 1H), 6.59 (dd, J=2.0, 8.2 Hz, 1H), 4.20 (s, 4H), 4.04 (s, 3H), 3.67 (s, 2H), 3.31-3.27 (m, 2H, obscured by water), 3.10-2.94 (m, 2H), 2.70-2.63 (m, 2H), 2.46-2.38 (m, 1H), 2.10-2.00 (m, 2H), 1.80-1.72 (m, 2H), 1.45-1.33 (m, 2H). ¹³CNMR (DMSO-d₆, 100 MHz) δ: 210.08, 162.01, 156.82 (d, J=254.1 Hz), 143.00, 142.00, 140.89 (d, J=7.1 Hz), 140.43, 138.09 (d, J=2.0 Hz), 137.88 (d, J=27.8 Hz), 127.68, 122.31, 118.10, 116.70, 115.31 (d, J=2.3 Hz), 64.00, 63.95, 56.63, 53.58, 52.25, 47.27, 45.85, 27.43, 20.55. HRMS (ESI) m/z calc'd for C₂₆H₂₉FN₃O₄ [M+H]⁺: 446.21366; found: 466.2128. UPLC: rt: 3.399 min, purity: 96.9%.
Example 24. 4-benzyl-6-(2-(1-(2-(3-fluoro-6-methoxy-1,5-naphthyridin-4-yl)ethyl)piperidin-4-yl)-2-oxoethyl)-2H-benzo[b][1,4]oxazin-3(4H)-one: Title compound was synthesized from piperidine 14c and aldehyde 2 using General Procedure 3. Purification by flash chromatography (compound eluted in 15% ethyl acetate in hexanes) and concentration in vacuo afforded the title compound as a yellow solid (50 mg, 0.104 mmol, 42% yield).

¹HNMR (CDCl₃, 400 MHz) δ: 8.61 (s, 1H), 8.17 (d, J=9.0 Hz, 1H), 7.84 (s, 1H), 7.07 (d, J=9.0 Hz, 1H), 6.92 (d, J=8.2 Hz, 1H), 6.77 (dd, J=2.0, 8.2 Hz, 1H), 6.62 (d, J=1.9 Hz, 1H), 4.60 (s, 2H), 4.07 (s, 3H), 3.67 (s, 2H), 3.41-3.35 (m, 2H), 3.09 (dt, J=3.5, 11.9 Hz, 2H), 2.77-2.71 (m, 2H), 2.42 (tt, J=4.0, 11.5 Hz, 1H), 2.15 (td, J=2.5, 11.5 Hz, 2H), 1.88-1.81 (m, 2H), 1.71 (qd, J=3.6, 11.6 Hz, 2H). ¹³CNMR (CDCl₃, 100 MHz) δ: 209.89, 165.40, 162.55, 157.45 (d, J=255.1 Hz), 142.85, 141.78 (d, J=6.8 Hz), 140.34, 138.67 (d, J=1.9 Hz), 138.06 (d, J=28.1 Hz), 130.61 (d, J=12.7 Hz), 128.86, 126.34, 125.32, 117.00 (d, J=29.8), 115.35 (d, J=2.75 Hz), 67.52, 57.33, 53.91, 52.97, 48.57, 46.56, 28.09, 21.14. HRMS (ESI) m/z calc'd for C₂₆H₂₈FN₄O₄ [M+H]⁺: 479.20891; found: 479.20825. UPLC: rt: 3.115 min, purity: 90.5%.

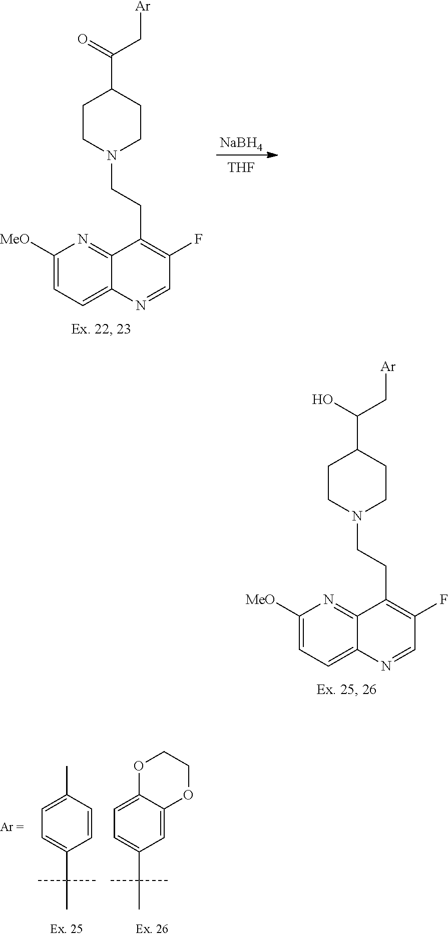

Example 25. 1-(1-(2-(3-fluoro-6-methoxy-1,5-naphthyridin-4-yl)ethyl)piperidin-4-yl)-2-(p-tolyl)ethan-1-ol: Title compound was synthesized from Example 22 using General Procedure 4. Purification by flash chromatography (compound eluted in 0.5% methanol in DCM) and concentration in vacuo afforded the title compound as a clear oil (23 mg, 0.054 mmol, 15% yield).

¹HNMR (DMSO-d₆, 400 MHz, contaminated w/ DMSO) δ: 8.76 (s, 1H), 8.27 (d, J=9.0 Hz, 1H), 7.23 (d, J=9.0 Hz, 1H), 7.08 (d, J=8.3 Hz, 2H), 7.04 (d, J=8.1 Hz, 2H), 4.33 (d, J=5.9 Hz, 1H), 4.03 (s, 3H), 3.41-3.35 (m, 1H), 3.30-3.26 (m, 3H), 3.05-2.97 (m, 2H), 2.68-2.61 (m, 3H), 2.25 (s, 3H), 1.96-1.87 (m, 2H), 1.73-1.67 (m, 1H), 1.53-1.47 (m, 1H), 1.33 (qd, J=3.8, 12.2 Hz, 1H), 1.24 (dd, J=3.5, 12.0 Hz, 1H), 1.20-1.11 (m, 1H). ¹³CNMR (DMSO-d₆, 100 MHz) δ: 161.96, 156.82 (d, J=254.1 Hz), 140.91 (d, J=7.1 Hz), 140.40, 138.07 (d, J=1.9 Hz), 137.98, 137.71, 136.93, 134.35, 130.20, 130.07, 129.13, 128.48, 115.26 (d, J=2.4 Hz), 74.66, 56.85, 53.53, 53.20, 28.59, 26.52, 20.68, 20.59. HRMS (ESI) m/z calc'd for C₂₅H₃₁FN₃O₂ [M+H]⁺: 424.23948; found: 424.23859. UPLC: rt: 3.519 min, purity: 90.3%.
Example 26. 2-(2,3-dihydrobenzo[b][1,4]dioxin-6-yl)-1-(1-(2-(3-fluoro-6-methoxy-1,5-naphthyridin-4-yl)ethyl)piperidin-4-yl)ethan-1-ol: Title compound was synthesized from Example 23 using General Procedure 4. Purification by flash chromatography (compound eluted in 0.5% methanol in DCM) and concentration in vacuo afforded the title compound as a white solid (5 mg, 0.011 mmol, 10% yield).

¹HNMR (DMSO-d₆, 400 MHz) δ: 8.77 (s, 1H), 8.28 (d, J=9.0 Hz, 1H), 7.24 (d, J=9.0 Hz, 1H), 6.71 (d, J=8.2 Hz, 1H), 6.69 (d, J=1.8 Hz, 1H), 6.63 (dd, J=1.9, 8.2 Hz, 1H), 4.35-4.30 (m, 1H), 4.19 (s, 4H), 4.04 (s, 3H), 3.38-3.28 (m, 2H, obscured by water), 3.09-2.98 (m, 2H), 2.7-2.61 (m, 2H), 2.59 (dd, J=3.7, 11.2 Hz, 1H), 2.41 (dd, J=8.1, 13.7 Hz, 1H), 2.00-1.88 (m, 2H), 1.74-1.67 (m, 1H), 1.55-1.47 (m, 1H), 1.39-1.31 (m, 4H). ¹³CNMR (DMSO-d₆, 100 MHz) δ: 162.00, 156.83 (d, J=254.2 Hz), 142.78, 141.36, 140.92 (d, J=6.9 Hz), 140.43, 138.10 (d, J=1.9 Hz), 137.89 (d, J=27.6 Hz), 133.04, 121.96, 117.71, 116.37, 115.32 (d, J=1.8 Hz), 74.59, 64.01, 63.93, 56.82, 53.59, 53.14, 28.48, 26.46, 20.57. HRMS (ESI) m/z calc'd for C₂₆H₃₁FN₃O₄ [M+H]⁺: 468.22931; found: 468.22876. UPLC: rt: 3.322 min, purity: 97.7%.

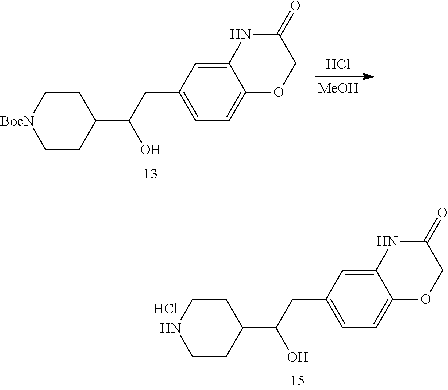 [SMILES: "MeOH" ; CC(C)(C)OC(=O)N1CCC(C(O)Cc2ccc3c(c2)NC(=O)CO3)CC1 ; Cl ; Cl ; O=C1COc2ccc(CC(O)C3CCNCC3)cc2N1]

6-(2-hydroxy-2-(piperidin-4-yl)ethyl)-2H-benzo[b][1,4]oxazin-3(4H)-one hydrochloride (15): Title compound was synthesized from piperidine 13 using General Procedure 2. No purification or isolation attempted. Crude compounds used directly in next step.

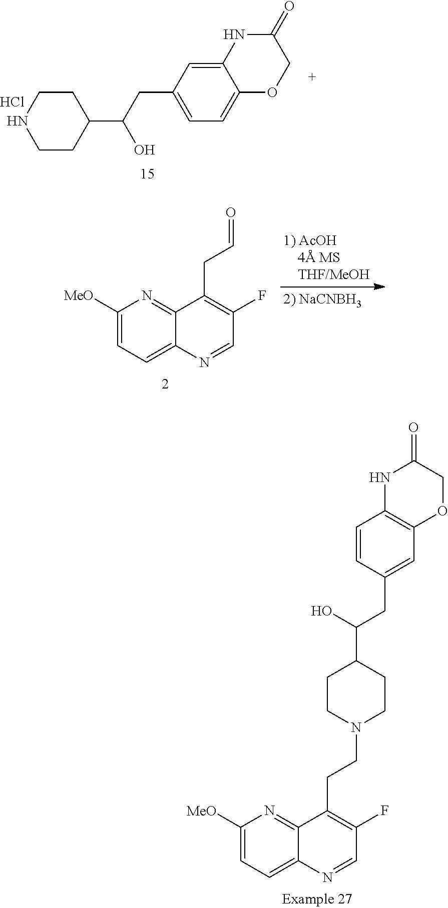 [SMILES: Cl ; O=C1COc2ccc(CC(O)C3CCNCC3)cc2N1 ; COc1ccc2ncc(F)c(CC=O)c2n1 ; COc1ccc2ncc(F)c(CCN3CCC(C(O)Cc4ccc5c(c4)OCC(=O)N5)CC3)c2n1]

Example 27. 7-(2-(1-(2-(3-fluoro-6-methoxy-1,5-naphthyridin-4-yl)ethyl)piperidin-4-yl)-2-hydroxyethyl)-2H-benzo[b][1,4]oxazin-3(4H)-one: Title compound was synthesized from piperidine 15 and aldehyde 2 using General Procedure 3. Purification by flash chromatography (compound eluted in 1% methanol in DCM) and concentration in vacuo afforded the title compound as a white solid (7 mg, 0.015 mmol, 21% yield).

¹HNMR (DMSO-d₆, 400 MHz, contaminated w/ DCM and DMSO) δ: 8.62 (s, 1H), 8.17 (d, J=9.0 Hz, 1H), 7.43 (s, 1H), 7.07 (d, J=9.0 Hz, 1H), 6.93 (d, J=8.2 Hz, 1H), 6.83 (dd, J=1.9, 8.2 Hz, 1H), 6.65 (d, J=1.8 Hz, 1H), 4.60 (s, 2H), 4.09 (s, 3H), 3.60-3.54 (m, 1H), 3.45-3.39 (m, 2H), 3.21-3.12 (m, 2H), 2.84 (dd, J=2.8, 13.8 Hz, 1H), 2.78-2.71 (m, 2H), 2.52 (dd, J=9.6, 13.8 Hz, 1H), 2.16-2.06 (m, 2H), 1.93-1.87 (m, 1H), 1.77-1.71 (m, 1H), 1.56-1.40 (m, 4H, obscured by water). ¹³CNMR (DMSO-d₆, 100 MHz, contaminated w/ DMSO) δ: 165.00, 161.97, 156.83 (d, J=254.0 Hz), 141.32, 140.92 (d, J=7.1 Hz), 140.41, 138.08 (d, J=1.8 Hz), 137.87 (d, J=27.7 Hz), 134.30, 126.70, 123.76, 116.66, 115.53, 115.28 (d, J=1.7 Hz), 74.65, 66.77, 56.92, 56.85, 53.55, 53.16, 30.17, 28.53, 28.01, 26.66, 20.67. HRMS (ESI) m/z calc'd for C₂₆H₃₀FN₄O₄ [M+H]⁺: 481.22456; found: 481.22397. UPLC: rt: 3.069 min, purity: 96.5%.

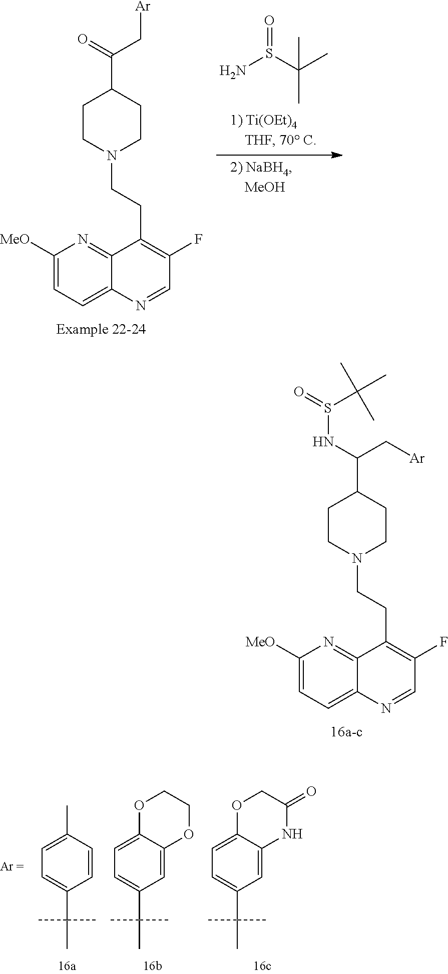

N-(1-(1-(2-(3-fluoro-6-methoxy-1,5-naphthyridin-4-yl)ethyl)piperidin-4-yl)-2-(p-tolyl)ethyl)-2-methylpropane-2-sulfinamide (16a): Title compound was synthesized from Example 22 using General Procedure 5. No purification or isolation attempted. Crude compounds used directly in next step.

N-(2-(2,3-dihydrobenzo[b][1,4]dioxin-6-yl)-1-(1-(2-(3-fluoro-6-methoxy-1,5-naphthyridin-4-yl)ethyl)piperidin-4-yl)ethyl)-2-methylpropane-2-sulfinamide (16b): Title compound was synthesized from Example 23 using General Procedure 5. No purification or isolation attempted. Crude compounds used directly in next step.

N-(1-(1-(2-(3-fluoro-6-methoxy-1,5-naphthyridin-4-yl)ethyl)piperidin-4-yl)-2-(3-oxo-3,4-dihydro-2H-benzo[b][1,4]oxazin-6-yl)ethyl)-2-methylpropane-2-sulfinamide (16c): Title compound was synthesized from Example 24 using General Procedure 5. No purification or isolation attempted. Crude compounds used directly in next step.

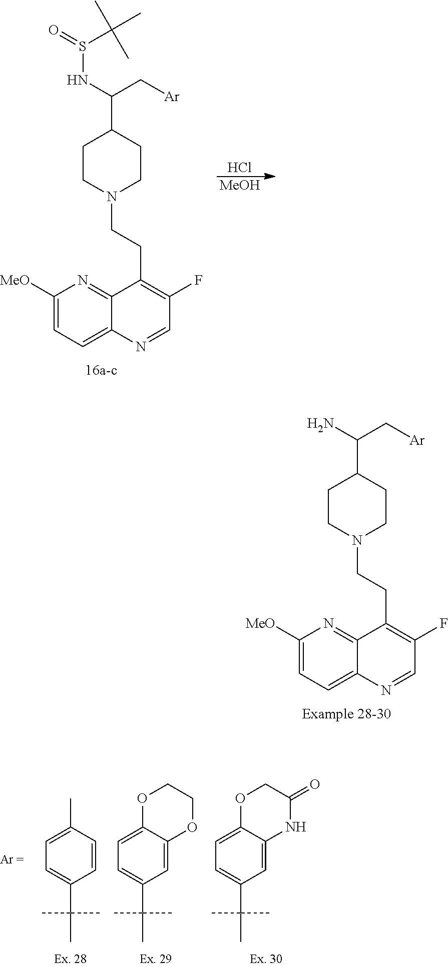

Example 28. 1-(1-(2-(3-fluoro-6-methoxy-1,5-naphthyridin-4-yl)ethyl)piperidin-4-yl)-2-(p-tolyl)ethan-1-amine: Title compound was synthesized from sulfinamide 16a using General Procedure 6. Purification by flash chromatography (compound eluted in 1% methanol in DCM) and concentration in vacuo afforded the title compound as a white solid (8 mg, 0.0189 mmol, 25% yield).

¹HNMR (DMSO-d₆, 400 MHz, contaminated w/ methanol) δ: 8.77 (s, 1H), 8.28 (d, J=9.0 Hz, 1H), 7.24 (d, J=9.0 Hz, 1H), 7.12 (s, 4H), 4.04 (s, 3H), 3.33-3.26 (m, 3H), 3.09-3.00 (m, 3H), 2.78 (dd, J=6.4, 13.9 Hz, 1H), 2.69-2.62 (m, 3H), 2.27 (s, 3H), 1.95-1.86 (m, 2H), 1.69-1.63 (m, 1H), 1.59-1.55 (m, 1H), 1.43-1.18 (m, 4H). ¹³CNMR (DMSO-d₆, 100 MHz, contaminated w/ chloroform) δ: 162.38, 156.67 (d, J=256.7 Hz), 140.55 (d, J=6.3 Hz), 140.49, 138.23 (d, J=10.9 Hz), 138.09 (d, J=14.5 Hz), 135.87, 133.28, 129.29, 129.22, 126.31 (d, J=12.6 Hz), 115.67 (d, J=2.3 Hz), 55.00, 54.21, 53.66, 51.04 (d, J=10.4 Hz), 35.16, 34.53, 24.74, 24.44, 20.64, 18.02. HRMS (ESI) m/z calc'd for C₂₅H₃₂FN₄O [M+H]⁺: 423.25547; found: 423.25473. UPLC: rt: 2.824 min, purity: 95.9%.
Example 29. 2-(2,3-dihydrobenzo[b][1,4]dioxin-6-yl)-1-(1-(2-(3-fluoro-6-methoxy-1,5-naphthyridin-4-yl)ethyl)piperidin-4-yl)ethan-1-amine: Title compound was synthesized from sulfinamide 16b using General Procedure 6. Purification by flash chromatography (compound eluted in 0.6% methanol in DCM) and concentration in vacuo afforded the title compound as an off-white solid (8 mg, 0.0171 mmol, 33% yield).

¹HNMR (DMSO-d₆, 400 MHz, contaminated with DMSO) δ: 8.77 (s, 1H), 8.28 (d, J=9.0 Hz, 1H), 7.24 (d, J=9.0 Hz, 1H), 6.76 (d, J=8.2 Hz, 1H), 6.72 (d, J=1.9 Hz, 1H), 6.66 (dd, J=2.0, 8.2 Hz, 1H), 4.98 (br s, 2H), 4.20 (s, 4H), 4.04 (s, 3H), 3.37-3.26 (m, 2H), 3.08-3.00 (m, 2H), 2.90-2.83 (m, 1H), 2.71-2.63 (m, 3H), 2.47-2.40 (m, 1H), 1.98-1.88 (m, 2H), 1.69-1.63 (m, 1H), 1.60-1.54 (m, 1H), 1.40-1.18 (m, 3H). ¹³CNMR (DMSO-d&, 100 MHz, contaminated with chloroform and DMSO) δ: 161.98, 156.84 (d, J=254.09), 143.09, 141.81, 140.92 (d, J=7.1 Hz), 140.43, 138.09 (d, J=1.8 Hz), 137.87 (d, J=27.7 Hz), 131.55, 130.09 (d, J=13.0 Hz), 121.89, 117.63, 116.81, 115.30 (d, J=2.4 Hz), 63.97 (d, J=8.3 Hz), 56.74, 56.22, 53.55, 53.09 (d, J=3.5 Hz), 37.63, 28.21, 26.70, 20.69. HRMS (ESI) m/z calc'd for C₂₆H₃₂FN₄O₃ [M+H]⁺: 467.24530; found: 467.24423. UPLC: rt: 2.735 min, purity: 98.2%.
Example 30. 6-(2-amino-2-(1-(2-(3-fluoro-6-methoxy-1,5-naphthyridin-4-yl)ethyl)piperidin-4-yl)ethyl)-2H-benzo[b][1,4]oxazin-3(4H)-one: Title compound was synthesized from sulfinamide 16c using General Procedure 6. Purification by flash chromatography (compound eluted in 1% methanol in DCM) and concentration in vacuo afforded the title compound as a white solid (5 mg, 0.0104 mmol, 17% yield).

¹HNMR (DMSO-d₆, 400 MHz) δ: 10.61 (s, 1H), 8.77 (s, 1H), 8.28 (d, J=9.0 Hz, 1H), 7.24 (d, J=9.0 Hz, 1H), 6.84 (d, J=8.2 Hz, 1H), 6.74-6.70 (m, 2H), 4.51 (s, 2H), 4.04 (s, 3H), 3.33-3.26 (m, 1H, obscured by water), 3.06-2.99 (m, 2H), 2.68-2.54 (m, 4H), 2.24 (dd, J=8.4, 12.9 Hz, 1H), 2.00-1.90 (m, 2H), 1.70-1.63 (m, 1H), 1.60-1.52 (m, 1H), 1.37-1.09 (m, 5H). ¹³CNMR (DMSO-d₆, 100 MHz, contaminated w/ DMSO) δ: 164.98, 161.98, 156.84 (d, J=255.3 Hz), 141.39, 140.92 (d, J=7.0 Hz), 140.42, 138.06 (d, J=7.5 Hz), 137.74, 134.56, 130.20, 130.07, 126.92, 123.60, 116.30, 115.75, 115.30, 66.75, 56.88, 56.62, 53.55, 53.34 (d, J=3.8 Hz), 28.81, 26.85, 20.72. HRMS (ESI) m/z calc'd for C₂₆H₃₁FN₅O₃ [M+H]⁺: 480.24054; found: 480.24045. UPLC: rt: 2.600 min, purity: 97.9%.

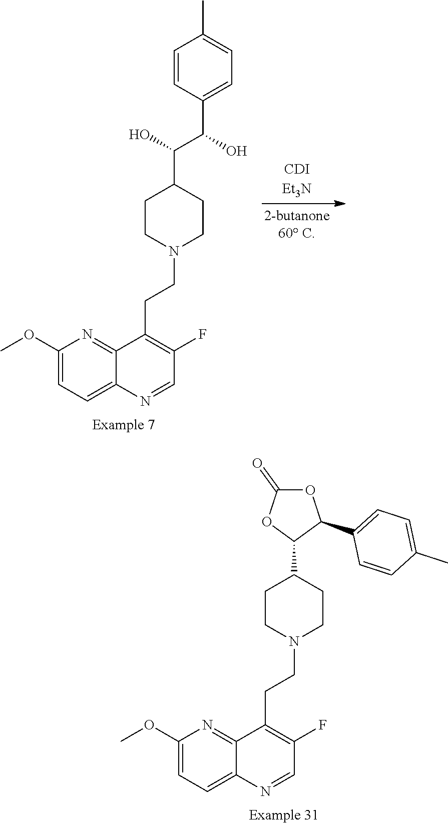

Example 31. (4S,5S)-4-(1-(2-(3-fluoro-6-methoxy-1,5-naphthyridin-4-yl)ethyl)piperidin-4-yl)-5-(p-tolyl)-1,3-dioxolan-2-one: Title compound was synthesized from Example 7 using General Procedure 16. Purification by flash chromatography (compound eluted in 2% methanol in DCM) and concentration in vacuo afforded the title compound as a clear oil (11 mg, 0.0236 mmol, 21% yield).

¹HNMR (CDCl₃, 400 MHz, contaminated w/ ethyl acetate) δ: 8.61 (s, 1H), 8.17 (d, J=9.0 Hz, 1H), 7.23 (s, 4H), 7.07 (d, J=9.0 Hz, 1H), 5.29 (d, J=6.2 Hz, 1H), 4.38 (t, J=6.7 Hz, 1H), 4.07 (s, 3H), 3.45-3.33 (m, 2H), 3.21-3.05 (m, 2H), 2.82-2.70 (m, 2H), 2.38 (s, 3H), 2.19-2.07 (m, 2H), 1.94-1.86 (m, 1H), 1.83-1.72 (m, 1H), 1.70-1.60 (m, 2H), 1.53-1.42 (m, 1H), 1.42-1.28 (m, 1H).

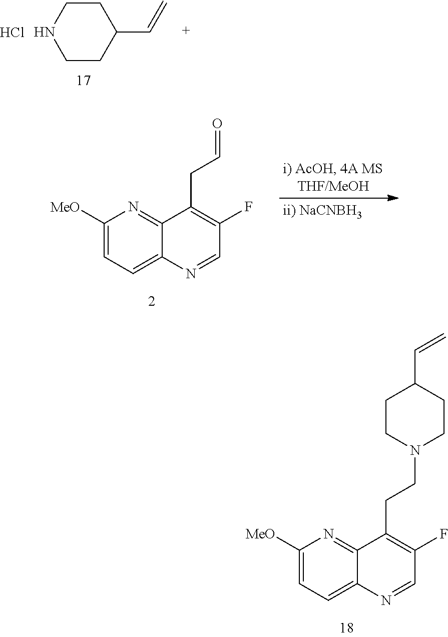

7-fluoro-2-methoxy-8-(2-(4-vinylpiperidin-1-yl)ethyl)-1,5-naphthyridine (18).: Prepared from amine 17 and 1.36 mmol aldehyde 2 according to General Procedure 3. Purification by flash chromatography (compound eluted in 10% ethyl acetate in hexanes) and concentration in vacuo afforded the title compound (290 mg, 0.919 mmol, 67% yield) as a clear oil. ¹HNMR (CDCl₃, 400 MHz) δ: 8.61 (s, 1H), 8.17 (d, J=9.0 Hz, 1H), 7.07 (d, J=9.1 Hz, 1H), 5.80 (ddd, J=17.3, 10.4, 6.4 Hz, 1H), 5.02 (br d, J=17.3 Hz, 1H), 4.96 (br d, J=10.3 Hz, 1H), 4.09 (s, 3H), 3.54-3.37 (m, 2H), 3.23-3.02 (m, 2H), 2.88-2.67 (m, 2H), 2.30-2.09 (m, 2H), 2.07-1.93 (m, 1H), 1.81-1.68 (m, 2H), 1.63-1.50 (m, 2H, overlapping with water).

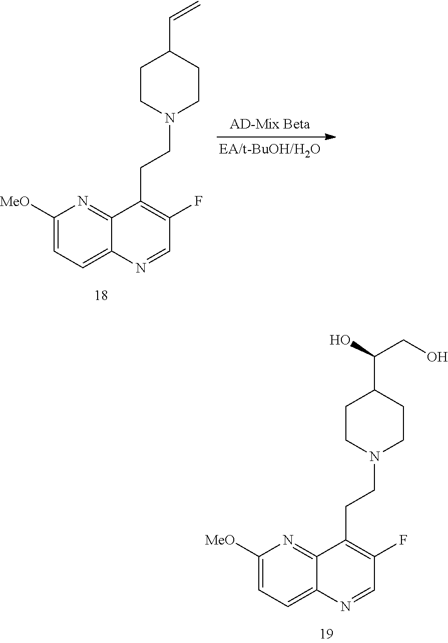 [SMILES: C=CC1CCN(CCc2c(F)cnc3ccc(OC)nc23)CC1 ; COc1ccc2ncc(F)c(CCN3CCC([C@@H](O)CO)CC3)c2n1]

(R)-1-(1-(2-(3-fluoro-6-methoxy-1,5-naphthyridin-4-yl)ethyl)piperidin-4-yl)ethane-1,2-diol (19). Prepared from alkene 18 and AD-Mix Beta according to General Procedure 17. Purification by flash chromatography (compound eluted in 60% ethyl acetate in hexanes) and concentration in vacuo afforded the title compound (290 mg, 0.83 mmol, 87% yield) as a slightly yellow oil, contaminated with dichloromethane.

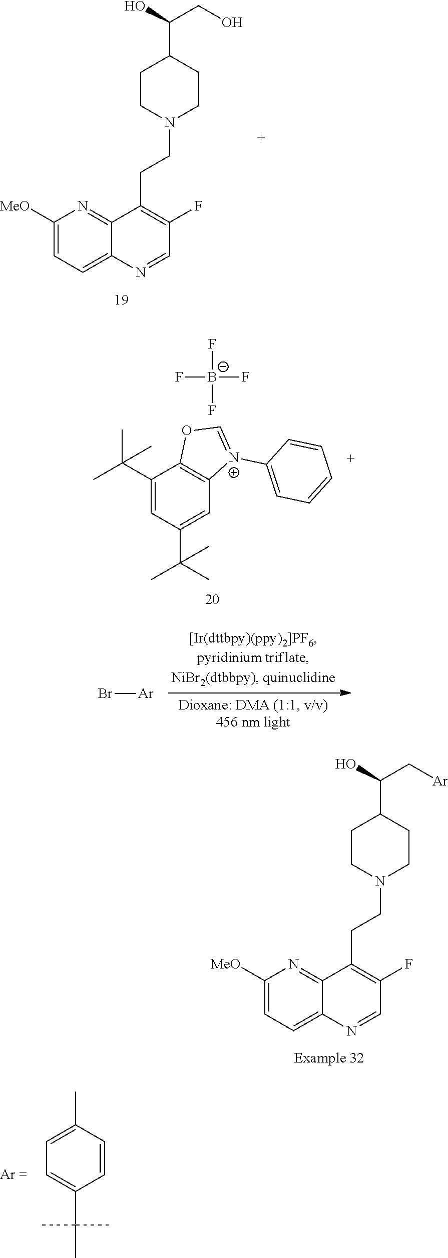

Example 32. (S)-1-(1-(2-(3-fluoro-6-methoxy-1,5-naphthyridin-4-yl)ethyl)piperidin-4-yl)-2-(p-tolyl)ethan-1-ol: A crude sample of the title compound was synthesized using diol 19, carbene precursor 20, and 4-bromotoluene via General Procedure 8. LC-LRMS rt: 3.754 min, m/z calc'd for C₂₅H₃₁FN₃O₂ [M+H]⁺: 424.240030; found: 424.4.

### Antibacterial Activity

Minimum inhibitory concentrations (MICs) were determined in triplicate according to Clinical and Laboratory Standards Institute (CLSI) guidelines using one or more strains of S. aureus or selected Gram-negative bacteria. Strain ATCC 29213 was used for initial evaluation, and ciprofloxacin, gepotidacin, and vancomycin were employed as positive controls in each assay. Compounds were also assayed using a USA300 (Hidron, A. I., et al., Emergence of Community-Acquired Meticillin-Resistant Staphylococcus aureus Strain USA300 as a Cause of Necrotising Community-Onset Pneumonia. Lancet Infect. Dis. 2009, 9. 384-392) isolate of MRSA as well as a S. aureus strain isolated from a cystic fibrosis patient.

Due to their novel binding mode, NBTIs are expected to preserve activity against fluoroquinolone-resistant strains. This assumption was evaluated for this series of NBTIs: the MICs against the ciprofloxacin-resistant USA300 strain were essentially identical to those against the ciprofloxacin-sensitive ATCC strain. The compounds also demonstrated equivalent MICs against a S. aureus isolate from a CF patient, further demonstrating their promising activity.

TABLE 1
MICs (μg/mL) of NBTIs against Staphylococcus aureus

| Example | ATCC 29213 | USA300^{a} | CF isolate^{b} | D83N^{c} | M121K^{c} |
|---|---|---|---|---|---|
| 1 | ≤0.25-0.5 | 0.5-2 | 0.5-2 | 8->32 | 2-4 |
| 2 | 0.25 | 0.5 | 0.5 | 2 | 2 |
| 3 | 0.5 | ≤0.25 | 1 | 16 | 16 |
| 4 | 0.5-1 | 1-2 | 2 | 16->32 | 16->32 |
| 5 | 1-4 | 1-4 | 1-8 | 16->32 | 16->32 |
| 6 | 8 | 8 | 16 | >32 | >32 |
| 7 | 1-2 | 2-4 | 2-4 | >32 | >32 |
| 8 | 1 | 2 | 2 | >32 | 32 |
| 9 | 1 | 1 | 4 | >32 | >32 |
| 10 | 4-8 | 4-8 | 4-8 | 32 | 32 |
| 11 | 8 | 8 | 9 | >16 | >16 |
| 12 | >64 | >64 | >64 | >64 | >64 |
| 13 | 0.125 | 0.25-0.5 | 0.25 | >32 | 16 |
| 14 | 0.03 | 0.03 | 0.03 | 8 | 4 |
| 15 | 2 | 2 | 1 | >64 | >64 |
| 16 | 0.03 | 0.06 | 0.125 | 16 | 4 |
| 17 | ≤0.125 | 0.25 | 0.25 | 16 | 8 |
| 18 | 2 | 4 | 1 | 64 | 64 |
| 19 | 0.5 | 0.5 | 1 | 4 | >16 |
| 20 | 0.5 | 1 | 1 | 8 | >8 |
| 21 | 1 | 0.5 | 2 | 32 | 64 |
| 22 | 0.125 | 0.125-0.5 | 0.125-0.5 | 4 | 8 |
| 23 | 4 | 4 | 8 | 32 | >32 |
| 24 | ≤0.125 | 0.25 | 0.25 | 16 | 32 |
| 25 | 0.06 | 0.25-0.5 | 0.25-0.5 | 8 | 8 |
| 26 | 0.25 | 0.25 | 0.25 | 4 | 4 |
| 27 | ≤0.125 | ≤0.125 | ≤0.125 | 16 | 32 |
| 28 | 1 | 4 | 2 | 32 | >32 |
| 29 | 2 | 4 | 4 | >16 | >16 |
| 30 | 16 | 4 | 8 | 64 | >64 |
| 31 | 2 | 2 | 4 | >32 | >32 |
| 32 | Not tested | Not tested | Not tested | Not tested | Not tested |
| gepotidacin | 0.25-1 | 0.125-1 | 0.125-0.5 | 4-32 | 8-32 |
| ciprofloxacin | 0.125-0.5 | 32 | 0.125-0.5 | 0.25-1 | 0.125-1 |
| vancomycin | 1-2 | 1 | 1 | 1 | 1 |

^{a}MRSA isolate.
^{b}Isolate from individual with cystic fibrosis.
^{c}29213-derived strain with amino acid substitution in GyrA domain of S. aureus DNA gyrase.

TABLE 2
MICs (μg/mL) of NBTIs against Representative Gram-negative pathogens

| | Escherichia | Acinetobacter | Pseudomonas | Salmonella | Salmonella |
|---|---|---|---|---|---|
| | coli | baumanii | aeruginosa | enterica | enterica |
| | ATCC | ATCC | ATCC | ATCC | 14028 |
| Example | 25922 | 19606 | 27853 | 14028 | ΔtolC^{a} |
| 1 | >64 | 64 | >64 | >64 | 0.5-1 |
| 2 | 8 | 8 | 64 | 64 | 0.125 |
| 3 | 4 | 8 | 4 | 16 | 0.25 |
| 4 | 16-64 | 16-64 | 64->64 | 64->64 | 0.5-1 |
| 5 | 16 | 8-16 | 64 | 64 | 0.5 |
| 6 | 16 | 32 | 64 | 64 | 1 |
| 7 | 64 | 32-64 | >64 | >64 | 1-2 |
| 8 | 32 | 16 | >64 | 64 | 1 |
| 9 | 4 | 16 | 8 | 32 | 0.25 |
| 10 | >64 | >64 | >64 | >64 | 8 |
| 11 | >64 | 64 | 64 | >64 | 4 |
| 12 | 64 | 32 | 64 | 64 | 16 |
| 13 | 32 | 16 | >64 | 64 | 1 |
| 14 | 8 | 4 | 64 | 32 | 0.125 |
| 15 | 8 | 64 | 64 | 32 | 0.5 |
| 16 | 16 | 16 | 64 | 64 | 1 |
| 17 | 16 | 8 | 64 | 32 | 0.25 |
| 18 | 8 | 32 | 32 | >64 | 8 |
| 19 | 64 | 64 | >64 | >64 | 1 |
| 20 | 16 | 32 | >64 | 64 | 0.25 |
| 21 | 4 | 8 | 32 | 16 | 0.5 |
| 22 | 16 | 32 | >64 | 64 | 0.5 |
| 23 | 64 | 64 | >64 | >64 | 1->1 |
| 24 | 2 | 4 | 8 | 16 | ≤0.125 |
| 25 | 16 | 32 | >64 | 64 | 1 |
| 26 | 8 | 8 | 64 | 32 | 0.25 |
| 27 | 4 | 4 | 16 | 16 | ≤0.125 |
| 28 | 16 | 16 | 64 | 64 | 4 |
| 29 | 8 | 16 | 64 | 32 | 2 |
| 30 | 8 | 32 | 64 | 32 | 4 |
| 31 | >64 | 64 | >64 | >64 | 8 |
| 32 | Not tested | Not tested | Not tested | Not tested | Not tested |
| gepotidacin | 0.5-2 | 32-64 | 2-16 | 2-8 | 0.125-0.5 |
| ciprofloxacin | 0.0075-0.03 | 1-8 | 0.25-2 | 0.015-0.03 | 0.00375-0.0075 |

^{a}Efflux-deficient strain.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A compound having Formula I: 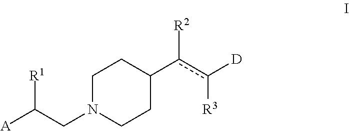
wherein,
the dashed line represents a bond that is present or absent, and when the bond is present, R² and R³ are both H;
A is a fused bicyclic aryl or bicyclic heteroaryl ring optionally substituted with C₁-C₂₄ alkyl, C₁-C₂₄ haloalkyl, C₁-C₂₄ alkoxy, C₂-C₂₄ alkenyl, C₂-C₂₄ alkynyl, C₅-C₁₅ aryl, C₄-C₁₅ heteroaryl, aldehyde, amino, amido, carboxylic acid, carboxylic ester, ether, carbamate, halo, hydroxy, oxo, cyano, nitro, silyl, sulfo-oxo, sulfonyl, sulfone, sulfoxide, or thiol; or A and R¹ together form a tricyclic ring optionally substituted with C₁-C₂₄ alkyl, C₁-C₂₄ haloalkyl, C₁-C₂₄ alkoxy, C₂-C₂₄ alkenyl, C₂-C₂₄ alkynyl, C₅-C₁₅ aryl, C₄-C₁₅ heteroaryl, aldehyde, amino, amido, carboxylic acid, carboxylic ester, ether, halo, hydroxy, oxo, cyano, nitro, silyl, sulfo-oxo, sulfonyl, sulfone, sulfoxide, or thiol;
D is an C₅-C₁₅ aryl or C₅-C₁₅ heteroaryl ring optionally substituted with C₁-C₂₄ alkyl, C₁-C₂₄ haloalkyl, C₁-C₂₄ alkoxy, C₂-C₂₄ alkenyl, C₂-C₂₄ alkynyl, C₅-C₁₅ aryl, C₄-C₁₅ heteroaryl, aldehyde, amino, amido, carboxylic acid, carboxylic ester, carbamate, ether, halo, hydroxy, oxo, nitro, silyl, sulfo-oxo, sulfonyl, sulfone, sulfoxide, or thiol;
R¹ is H, OH, or together with A forms a tricyclic ring optionally substituted with C₁-C₂₄ alkyl, C₁-C₂₄ haloalkyl, C₁-C₂₄ alkoxy, C₂-C₂₄ alkenyl, C₂-C₂₄ alkynyl, C₅-C₁₅ aryl, C₄-C₁₅ heteroaryl, aldehyde, amino, amido, carboxylic acid, carboxylic ester, ether, halo, hydroxy, oxo, cyano, nitro, silyl, sulfo-oxo, sulfonyl, sulfone, sulfoxide, or thiol;
R² and R³ are, independently, chosen from H, OH, Cl, F, Br, I, CN, NO₂, NH₂, CF₃, CO₂H, CO₂NH₂, CO₂NHR¹⁰, CO₂R¹⁰, C(O)R¹⁰, C(O)NH₂, C(O)NHR¹⁰, NHC(O)R¹⁰, NHSOR¹⁰, NH, SO₂R¹⁰, oxo, and C₁-C₆ alkyl or C₁-C₆ alkoxyl optionally substituted with C₁-C₂₄ alkoxy, C₂-C₂₄ alkenyl, C₂-C₂₄ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol; or together R² and R³ form a carbamate or carbonate;
R¹⁰ is H, C₁-C₆ alkyl, C₂-C₂₄ alkenyl, C₂-C₂₄ alkynyl, C₅-C₁₅ aryl, or C₄-C₁₅ heteroaryl;
or a pharmaceutically acceptable salt thereof.

2. The compound of claim 1, wherein R¹ is H or OH.

3. The compound of claim 1, wherein R² and R³ are, independently, chosen from H, OH, and NH₂.

4. The compound of claim 1, wherein R² is NH₂, H, or OH.

5. (canceled)

6. The compound of claim 1, wherein R³ is NH₂, H, or OH.

7. (canceled)

8. The compound of claim 1, wherein R² and R³ together form a carbamate or carbonate.

9. (canceled)

10. The compound of claim 1, wherein A is a fused bicyclic aryl or bicyclic heteroaryl ring having Formula II: 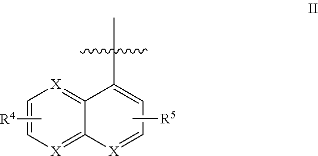
wherein,
each X is, independently, CH or N; and
R⁴ and R⁵ are, independently, chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with C₁-C₂₄ alkenyl, C₂-C₂₄ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol.

11. The compound of claim 10, wherein R⁴ and R⁵ are, independently, chosen from H, Cl, F, Br, I, CN, OH, and unsubstituted C₁-C₆ alkyl or C₁-C₆ alkoxyl.

12. (canceled)

13. The compound of claim 10, wherein R⁴ and R⁵ are, independently, chosen from F and methoxyl.

14. The compound of claim 10, wherein two X's are N and the other X is CH or two X's are CH and the other X is N.

15. (canceled)

16. The compound of claim 1, wherein A is a fused bicyclic aryl or bicyclic heteroaryl ring having Formula III: 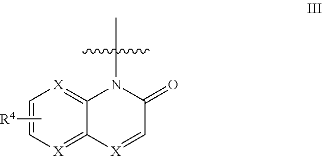
wherein,
each X is, independently, CH or N;
R⁴ is chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with C₁-C₂₄ alkenyl, C₂-C₂₄ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol.

17. The compound of claim 16, wherein R⁴ is chosen from H, Cl, F, Br, I, OH, and unsubstituted C₁-C₆ alkyl or C₁-C₆ alkoxyl.

18. (canceled)

19. The compound of claim 16, wherein R⁴ is chosen from F and methoxyl.

20. The compound of claim 1, wherein A and R¹ together have Formula IX, X, XI, or XII 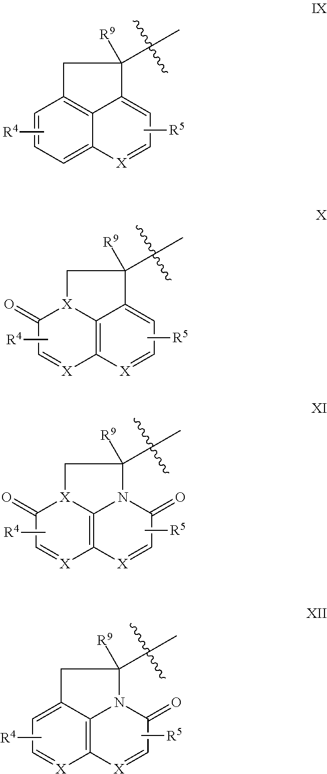
wherein,
each X is, independently, CH, N, or CR⁸; and
R⁴ and R⁵ are, independently, chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with C₁-C₂₄ alkenyl, C₂-C₂₄ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol;
each R⁸ is, independently, Cl, F, CN, OH, OCH₃, CH₃, or NH₂; and
R⁹ is H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, or C₁-C₆ alkyl or C₁-C₆ alkoxyl optionally substituted with C₁-C₂₄ alkyl, C₁-C₂₄ alkoxy, C₂-C₂₄ alkenyl, C₂-C₂₄ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol.

21. The compound of claim 20, wherein R⁴ is H and R⁵ is F.

22. The compound of claim 1, wherein D is aryl or heteroaryl ring having Formula IV-VIII or XIII: 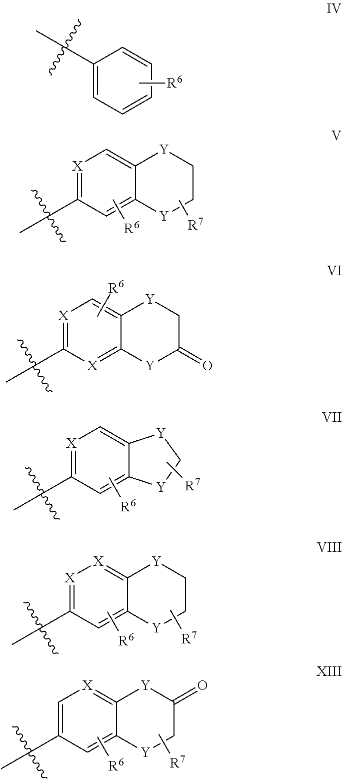
wherein,
each X is, independently, chosen from CH and N;
each Y is, independently, chosen from O, S, NH, and CH₂; and
R⁶ and R⁷ are, independently, chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with C₁-C₂₄ alkoxy, C₂-C₂₄ alkenyl, C₂-C₂₄ alkynyl, C₅-C₁₅ aryl, C₄-C₁₅ heteroaryl, aldehyde, amino, carboxylic acid, ester, ether, halo, hydroxy, oxo, cyano, nitro, silyl, sulfo-oxo, sulfonyl, sulfone, sulfoxide, or thiol.

23. The compound of claim 22, wherein R⁶ and R⁷ are, independently, chosen from H, Cl, F, Br, I, CN, OH, and unsubstituted C₁-C₆ alkyl or C₁-C₆ alkoxyl.

24. (canceled)

25. The compound of claim 22, wherein R⁶ and R⁷ are both H.

26. The compound of claim 22, wherein both Y are O.

27. The compound of claim 22, wherein one Y is S and the other is O.

28. The compound of claim 22, wherein one Y is NH and the other is O.

29. (canceled)

30. The compound of claim 1, wherein D is 4-methylphenyl.

31. The compound of claim 1, wherein A is

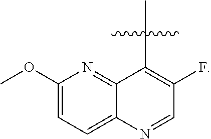 [SMILES: *c1c(F)cnc2ccc(OC)nc12]

32. (canceled)

33. (canceled)

34. (canceled)

35. A method of treating an infection in a subject, comprising administering to the subject an effective amount of the compound of claim 1.

36. The method of claim 35, wherein the infection is a Staphylococcus aureus infection.

37. The method of claim 35, wherein the infection is a methicillin-resistant S. aureus (MRSA) infection.
